(19) United States
(12) Patent Application Publication
(10) Pub. No.: US 2024/0366636 A1
(43) Pub. Date: Nov. 7, 2024

(54) ORAL CANNABINOID PHARMACEUTICAL COMPOSITIONS AND METHODS OF TREATING INFLAMMATORY GASTROINTESTINAL DISORDERS

(71) Applicant: Curio IP, LLC, Towson, MD (US)

(72) Inventors: Edward M. Rudnic, Georgetown, TX (US); Michael Bronfein, Palm Beach Gardens, FL (US); Neil David Goldberg, Baltimore, MD (US)

(21) Appl. No.: 18/759,282

(22) Filed: Jun. 28, 2024

Related U.S. Application Data
(63) Continuation of application No. 18/304,808, filed on Apr. 21, 2023, now abandoned.
(60) Provisional application No. 63/333,982, filed on Apr. 22, 2022.

(51) Int. Cl. A61K 31/00; A61K 9/00; A61K 9/20; A61K 9/48; A61P 1/04; A61P 1/12; A61P 29/00

(52) U.S. Cl. CPC A61K 31/658; A61K 9/0053; A61K 9/2054; A61K 9/4808; A61P 1/04; A61P 1/12; A61P 29/00

(57) ABSTRACT

Oral cannabinoid pharmaceutical compositions and methods of treating inflammatory gastrointestinal disorders using the oral cannabinoid pharmaceutical compositions are described.

### RELATED APPLICATION

This application is the continuation of U.S. application Ser. No. 18/304,808, filed Apr. 21, 2023, which claims the benefit of U.S. Provisional Application No. 63/333,982 filed Apr. 22, 2022, the contents of which are herein incorporated by reference in their entirety.

### TECHNICAL FIELD

The present disclosure relates to oral cannabinoid pharmaceutical compositions and methods of treating inflammatory gastrointestinal (GI) disorders using the oral cannabinoid pharmaceutical compositions.

### BACKGROUND

Inflammatory gastrointestinal disorders are a prevalent medical problem worldwide. For example, the Centers for Disease Control and Prevention estimates that approximately 1.3% of adults in the United States have been diagnosed with Inflammatory Bowel Disease (e.g., Crohn's disease or Ulcerative Colitis). However, development of new and effective therapeutics for treating inflammatory gastrointestinal disorders remains a significant challenge.

### SUMMARY

The present disclosure provides oral cannabinoid pharmaceutical compositions and methods of treating inflammatory gastrointestinal disorders using the oral cannabinoid pharmaceutical compositions.

According to a first aspect, the present disclosure relates to an oral pulse-release dosage form comprising a total dose of one or more cannabinoid active pharmaceutical ingredients (API), wherein the oral pulse-release dosage form comprises: a first pulse-release component (C₁) comprising a first portion (P₁) of the one or more cannabinoid API (API-P₁); and a second pulse-release component (C₂) comprising a second portion (P₂) of the one or more cannabinoid API (API-P₂); wherein the total dose of each of the one or more cannabinoid API is divided between the first portion (P₁) in the first pulse-release component (C₁) and the second portion (P₂) in the at least second pulse-release component (C₂); and wherein when the pulse-release dosage form is placed in an aqueous solution of 0.1N HCl for two hours, followed by phosphate buffered solution at pH 6.0 and 37° C.±0.5° C. for four hours, followed by phosphate buffered solution at pH 7.2 and 37° C.±0.5° C. for up to fourteen hours, the pulse-release dosage form provides release of the one or more cannabinoid API-P₂ beginning from 1 to 6 hours after release of the API-P₁ begins.

The oral pulse-release dosage form may include the following details, which can be combined with one another in any combinations unless clearly mutually exclusive:
(i) The oral pulse-release dosage form may further comprise a third pulse-release component (C₃) comprising a third portion (P₃) of the one or more cannabinoid API (API-P₃); wherein the total dose of each of the one or more cannabinoid API is further divided between the first portion (P₁) in the first pulse-release component (C₁), the second portion (P₂) in the second pulse-release component (C₂), and the third portion (P₃) in the third pulse-release component (C₃); and wherein when the pulse-release dosage form is placed in an aqueous solution of 0.1N HCl for two hours, followed by phosphate buffered solution at pH 6.0 and 37° C.±0.5° C. for four hours, followed by phosphate buffered solution at pH 7.2 and 37° C.±0.5° C. for up to fourteen hours, the pulse-release dosage form provides release of the one or more cannabinoid API-P₃ beginning from 1 to 6 hours after release of the API₁P₂ begins.
(ii) The oral pulse-release dosage form may further comprise a fourth pulse-release component (C₄) comprising a fourth portion (P₄) of the one or more cannabinoid API (API-P₄); wherein the total dose of each of the one or more cannabinoid API is further divided between the first portion (P₁) in the first pulse-release component (C₁), the second portion (P₂) in the second pulse-release component (C₂), the third portion (P₃) in the third pulse-release component (C₃), and the fourth portion (P₄) in the fourth pulse-release component (C₄); and wherein when the pulse-release dosage form is placed in an aqueous solution of 0.1N HCl for two hours, followed by phosphate buffered solution at pH 6.0 and 37° C.±0.5° C. for four hours, followed by phosphate buffered solution at pH 7.2 and 37° C.±0.5° C. for up to fourteen hours, the pulse-release dosage form provides release of the one or more cannabinoid API-P₄ beginning from 1 to 6 hours after release of the API-P₃ begins.
(iii) The oral pulse-release dosage form may further comprise a fifth pulse-release component (C₅) comprising a fifth portion (P₅) of the one or more cannabinoid API (API-P₅); wherein the total dose of each of the one or more cannabinoid API is further divided between the first portion (P₁) in the first pulse-release component (C₁), the second portion (P₂) in the second pulse-release component (C₂), the third portion (P₃) in the third pulse-release component (C₃), the fourth portion (P₄) in the fourth pulse-release component (C₄), and the fifth portion (P₅) in the fifth pulse-release component (C₅); and wherein when the pulse-release dosage form is placed in an aqueous solution of 0.1N HCl for two hours, followed by phosphate buffered solution at pH 6.0 and 37° C.±0.5° C. for four hours, followed by phosphate buffered solution at pH 7.2 and 37° C.±0.5° C. for up to fourteen hours, the pulse-release dosage form provides release of the one or more cannabinoid API-P₅ beginning from 1 to 6 hours after release of the API-P₄ begins.
(iv) The pulse-release dosage form may provide a second time of peak release rate (PRR₂) of each of the one or more cannabinoid API (PRR₂API) from about 1 to 6 hours after a first time of peak release rate (PRR₁) of each of the one or more cannabinoid API (PRR₁API).
(v) The PRR₁ may be after 1, 2, 3, 4, 5, 6, 7, or 8 hours.
(vi) The pulse-release dosage form may provide a third time of peak release rate (PRR₃) of each of the one or more cannabinoid API (PRR₃API) from about 1 to 6 hours after a second time of peak release rate (PRR₂) of each of the one or more cannabinoid API (PRR₂API).
(vii) The pulse-release dosage form may provide a fourth time of peak release rate (PRR₄) of each of the one or more cannabinoid API (PRR₄API) from about 1 to 6 hours after a third time of peak release rate (PRR₃) of each of the one or more cannabinoid API (PRR₃API).
(viii) The pulse-release dosage form may provide a fifth time of peak release rate (PRR₅) of each of the one or more cannabinoid API (PRR₅API) from about 1 to 6 hours after a fourth time of peak release rate (PRR₄) of each of the one or more cannabinoid API (PRR₄API).
(ix) The one or more cannabinoid API may comprise cannabigerol (CBG).
(x) The total dose of CBG may be up to 150 mg.
(xi) The one or more cannabinoid API may further comprise cannabidiol (CBD).
(xii) The total dose of CBD may be up to 40 mg.
(xiii) The one or more cannabinoid API may further comprise delta-9-tetrahydrocannabinol (THC).
(xiv) The total dose of THC may be up to 2 mg.
(xv) Release of the total dose of the one or more cannabinoid API from the oral pulse-release dosage form may occur in up to 20 hours, up to 19 hours, up to 18 hours, up to 17 hours, up to 16 hours, up to 15 hours, up to 14 hours, up to 13 hours, up to 12 hours, up to 11 hours, up to 10 hours, up to 9 hours, up to 8 hours, or less.
(xvi) The first pulse-release component (C₁) may be an immediate release (IR) formulation or a delayed-release (DR) formulation, wherein the first pulse-release component (C₁) comprises from 10% to 50% of the total dose independently of each of the one or more cannabinoid API; and the second pulse-release component (C₂) may be a delayed-release (DR) formulation comprising from 50% to 90% of the total dose independently of each of the one or more cannabinoid API.
(xvii) The first pulse-release component (C₁) may be an immediate release (IR) formulation or a delayed-release (DR) formulation, wherein the first pulse-release component (C₁) comprises from 10% to 30% of the total dose independently of each of the one or more cannabinoid API; and the second pulse-release component (C₂) and the third pulse-release component (C₃) may be delayed-release (DR) formulations, wherein the second pulse-release component (C₂) comprises from 20% to 40% of the total dose independently of each of the one or more cannabinoid API, and the third pulse-release component (C₃) comprises from 30% to 70% of the total dose independently of each of the one or more cannabinoid API, such that together the second pulse-release component (C₂) and the third pulse-release component (C₃) comprise from 70% to 90% of the total dose independently of each of the one or more cannabinoid API.
(xviii) The first pulse-release component (C₁) may be an immediate release (IR) formulation or a delayed-release (DR) formulation, wherein the first pulse-release component (C₁) comprises from 5% to 20% of the total dose independently of each of the one or more cannabinoid API; and the second pulse-release component (C₂), the third pulse-release component (C₃), and the fourth pulse-release component (C₄) may be delayed-release (DR) formulations, wherein the second pulse-release component (C₂) comprises from 15% to 30% of the total dose independently of each of the one or more cannabinoid API, the third pulse-release component (C₃) comprises from 20% to 40% of the total dose independently of each of the one or more cannabinoid API, and the fourth pulse-release component (C₄) comprises from 10% to 60% of the total dose independently of each of the one or more cannabinoid API, such that together the second pulse-release component (C₂), the third pulse-release component (C₃), and the fourth pulse-release component (C₄) comprise from 80% to 95% of the total dose independently of each of the one or more cannabinoid API.
(xix) The first pulse-release component (C₁) may be an immediate release (IR) formulation or a delayed-release (DR) formulation, wherein the first pulse-release component (C₁) comprises from 5% to 15% of the total dose independently of each of the one or more cannabinoid API; and the second pulse-release component (C₂), the third pulse-release component (C₃), the fourth pulse-release component (C₄), and the fifth pulse-release component (C₅) may be delayed-release (DR) formulations, wherein the second pulse-release component (C₂), comprises from 10% to 20% of the total dose independently of each of the one or more cannabinoid API, the third pulse-release component (C₃) comprises from 15% to 25% of the total dose independently of each of the one or more cannabinoid API, the fourth pulse-release component (C₄) comprises from 15% to 25% of the total dose independently of each of the one or more cannabinoid API, and the fifth pulse-release component (C₅) comprises from 15% to 55% of the total dose independently of each of the one or more cannabinoid API, such that together the second pulse-release component (C₂), the third pulse-release component (C₃), the fourth pulse-release component (C₄) and the fifth pulse-release component (C₅) comprise from 85% to 95% of the total dose independently of each of the one or more cannabinoid API.
(xx) The immediate release (IR) formulation may comprise one or more pharmaceutically-acceptable excipients.
(xxi) The one or more pharmaceutically-acceptable excipient may be selected from the group consisting of: microcrystalline cellulose, corn starch, pregelatinized starch, potato starch, rice starch, sodium carboxymethyl starch, hydroxypropylcellulose, hydroxypropylmethylcellulose, hydroxyethylcellulose, ethylcellulose, chitosan, hydroxychitosan, hydroxymethylatedchitosan, cross-linked chitosan, cross-linked hydroxymethyl chitosan, maltodextrin, mannitol, sorbitol, dextrose, maltose, fructose, glucose, levulose, sucrose, polyvinylpyrrolidone (PVP), an acrylic acid derivative (Carbopol, Eudragit, etc.), and a polyethylene glycol (PEG) such a low molecular weight PEGs (PEG2000-10000) and high molecular weight PEGs (Polyox) with molecular weights above 20,000 daltons, or any combination thereof.
(xxii) The pharmaceutically-acceptable excipients may comprise from 1% to 60% by weight of the immediate release (IR) formulation.
(xxiii) The immediate release (IR) formulation may further comprise one or more pharmaceutically-acceptable surfactants.
(xxiv) The one or more pharmaceutically-acceptable surfactant may be selected from the group consisting of sodium lauryl sulfate, sodium monoglycerate, sorbitan monooleate, polyoxyethylene sorbitan monooleate, glyceryl monostearate, glyceryl monooleate, glyceryl monobutyrate, a non-ionic surfactant such as the Pluronic line of surfactants, and any other material with surface active properties.
(xxv) The pharmaceutically-acceptable surfactants may comprise from 0.05% to 15% by weight of the immediate release (IR) formulation.
(xxvi) The delayed-release (DR) formulation may comprise a core comprising the one or more cannabinoid API and one or more pharmaceutically-acceptable excipients and optionally one or more pharmaceutically-acceptable surfactants; and delayed-release (DR) components comprising one or more polymers in an amount and configuration to delay release of the one or more cannabinoid API from the core.
(xxvii) The one or more polymers of the delayed-release (DR) components may be comprised in a layer coating the core, or integrated within the core, or both.
(xxviii) The delayed-release (DR) formulation may be a non-pH sensitive delayed-release formulation, a pH sensitive delayed-release formulation, or a combination thereof.
(xxix) The one or more polymers of the delayed-release (DR) components of the non-pH sensitive delayed-release formulation may comprise polyethylene glycol (PEG) with molecular weight above 4,000 daltons (Carbowax, Polyox), a wax such as white wax or bees wax, paraffin, an acrylic acid derivative (Eudragit), propylene glycol, ethylcellulose, or any combination thereof.
(xxx) The one or more polymers may comprise from 0.05% to 25% by weight of the delayed-release (DR) formulation.
(xxxi) The one or more polymers of the delayed-release (DR) components of the pH sensitive delayed-release formulation may comprise cellulose acetate pthalate, Eudragit L, a pthalate salt of a cellulose derivative, or any combination thereof.
(xxxii) The one or more polymers may comprise from 4% to 25% by weight of the delayed-release (DR) formulation.
(xxxiii) Each of the pulse-release components may be formulated as a plurality of particles or pellets.
(xxxiv) The particles or pellets may be contained in a capsule, formed as a tablet, or suspended in a liquid.
(xxxv) the first pulse-release component (C₁) may be an immediate release (IR) formulation or a delayed-release (DR) formulation, and the first pulse-release component (C₁) may comprise up to 10%, up to 20%, up to 30%, up to 40%, up to 50%, up to 60%, up to 70%, up to 80%, up to 90%, or up to 100% of the total dose independently of each of the one or more cannabinoid API; and the second pulse-release component (C₂) and the third pulse-release component (C₃) may be delayed-release (DR) formulations, wherein the second pulse-release component (C₂) may comprise up to 10%, up to 20%, up to 30%, up to 40%, up to 50%, up to 60%, up to 70%, up to 80%, up to 90%, or up to 100% of the total dose independently of each of the one or more cannabinoid API, and the third pulse-release component (C₃) may comprise up to 10%, up to 20%, up to 30%, up to 40%, up to 50%, up to 60%, up to 70%, up to 80%, up to 90%, or up to 100% of the total dose independently of each of the one or more cannabinoid API.
(xxxvi) the first pulse-release component (C₁) may comprise up to 90%, up to 91%, up to 92%, up to 93%, up to 94%, up to 95%, up to 96%, up to 97%, up to 98%, up to 99%, or up to 100% of a total THC dose; the third pulse-release component (C₃) may comprise up to 90%, up to 91%, up to 92%, up to 93%, up to 94%, up to 95%, up to 96%, up to 97%, up to 98%, up to 99%, or up to 100% of a total CBG dose; the second pulse-release component (C₂) may comprise up to 60%, up to 61%, up to 62%, up to 63%, up to 64%, up to 65%, up to 66%, up to 67%, up to 68%, up to 69%, or up to 70% % of a total CBD dose; and the third pulse-release component (C₃) may comprise up to 30%, up to 31%, up to 32%, up to 33%, up to 34%, up to 35%, up to 36%, up to 37%, up to 38%, up to 39%, or up to 40% of the total CBD dose.

According to a second aspect, the present disclosure relates to a method of treating an inflammatory gastrointestinal disorder in a subject, the method comprising orally administering a therapeutically effective amount of an oral pulse-release dosage form described herein to the subject.

The method may include the following details, which can be combined with one another in any combinations unless clearly mutually exclusive:
(i) The inflammatory gastrointestinal disorder may comprise inflammation of the upper gastrointestinal tract, the lower gastrointestinal tract, or both
(ii) The administering may be one, two, three or four times per day.
(iii) The inflammation may comprise inflammation of the upper gastrointestinal tract, and the first pulse-release component (C₁) may be an immediate release (IR) formulation.
(iv) The inflammation may comprise inflammation of the lower gastrointestinal tract, and the first pulse-release component (C₁) may be a delayed release (IR) formulation.
(v) The inflammatory gastrointestinal disorder may comprise irritable bowel syndrome, inflammatory bowel disease, Crohn's disease, ulcerative colitis, perianal fistulizing disease (e.g., ulcerative colitis-related or Crohn's disease-related), left sided colitis, ulcerative proctitis, Gastro-Esophogeal Reflux Disease (GERD), an inflammatory gastrointestinal disorder following Gall bladder removal, an undiagnosed inflammatory gastrointestinal disorder, or any combinations thereof.
(vi) The administering may result in treatment of the inflammatory gastrointestinal disorder within 6 hours post-dose, within 12 hours post-dose, within 24 hours post-dose, or greater than 24 hours post-dose.
(vii) The administering may result in treating one or more symptoms of the inflammatory gastrointestinal disorder for up to 1 hour per day, up to 2 hours per day, up to 3 hours per day, up to 4 hours per day, up to 5 hours per day, up to 6 hours per day, up to 12 hours per day, up to 24 hours per day, or for longer than 24 hours.
(viii) The one or more symptoms may comprise pain, bloating, diarrhea, loss of appetite, or any combinations thereof.

### BRIEF DESCRIPTION OF THE DRAWINGS

The present disclosure may be further understood through reference to the attached figures in combination with the detailed description that follows.

FIG. 1 is a graph reporting the responses from clinical study subjects to a survey question regarding treatment effects on gastrointestinal symptoms of twice-daily administration of an example pulse-release cannabinoid oral dosage form.

FIG. 2 is a graph reporting the responses from clinical study subjects to a survey question regarding onset of treatment effects on gastrointestinal symptoms of twice-daily administration of an example pulse-release cannabinoid oral dosage form.

FIG. 3 is a graph reporting the responses from clinical study subjects to a survey question regarding treatment effects on gastrointestinal symptoms in various conditions of twice-daily administration of an example pulse-release cannabinoid oral dosage form.

FIG. 4 is a graph reporting the responses from clinical study subjects to a survey question regarding how twice-daily administration of an example pulse-release cannabinoid oral dosage form made the subjects feel in general (positive results).

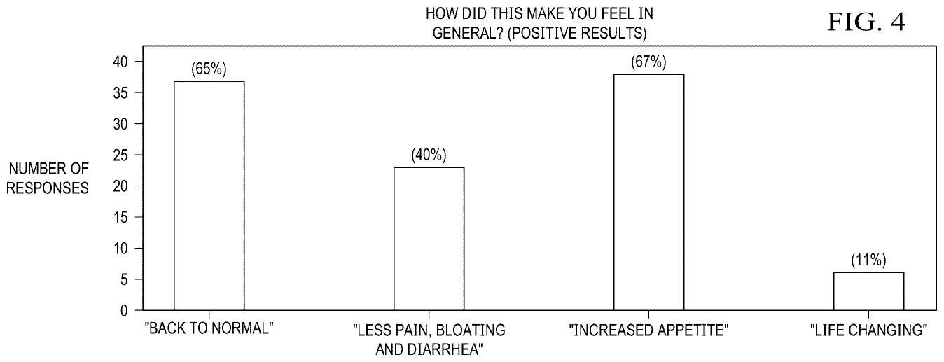

FIG. 5 is a graph reporting the responses from clinical study subjects to a survey question regarding how twice-daily administration of an example pulse-release cannabinoid oral dosage form made the subjects feel in general (negative results).

FIG. 6 is a graph reporting the responses from clinical study subjects to a survey question regarding whether the subjects would be inclined to take the example pulse-release cannabinoid oral dosage form again in future.

### DETAILED DESCRIPTION

The present disclosure relates in several embodiments described herein to oral cannabinoid pharmaceutical compositions and methods of treating inflammatory gastrointestinal disorders using the oral cannabinoid pharmaceutical compositions. In various embodiments, the inflammatory gastrointestinal disorders include, without limitation, Inflammatory Bowel Disease (IBD), Crohn's Disease (CD), and other inflammatory disorders of the gastrointestinal tract. In several embodiments, the pharmaceutical compositions described herein are capable of delivering one or more cannabinoid active pharmaceutical ingredients (API) locally to one or more inflamed regions of the gastrointestinal tract of a subject.

Cannabis sativa is one of the most widely consumed plants globally and has been used historically for medicinal purposes. It consists of approximately 100 phytochemicals, namely cannabinoids and multiple terpenes. Cannabinoids act on the CB1 and CB2 receptors as well as G protein-coupled receptor 55 (GPR55) and 5-hydroxytriptamine (5-HT) receptors of the endogenous endocannabinoid system in the gastrointestinal tract.

Crohn's disease is one example of an inflammatory disorder of the gastrointestinal tract. It is a form of Inflammatory Bowel Disease (IBD) accounting for 42% of IBD patients, and presents with the chronic symptoms of diarrhea, abdominal pain, weight loss, fatigue and occasionally rectal bleeding and is characterized by periods of crises along with periods of remission. Approved therapies for Crohn's disease include biologics of monoclonal antibodies to immune factors (e.g. anti-TNF, or anti-integrin), immunosuppressants and anti-inflammatories which target symptom improvement but can increase opportunistic infections, are costly, and are ineffective in some patients who either do not respond well or become refractory to antibody treatment.

Cannabinoids may decrease inflammation, improve abdominal spasms, cramps and visceral pain, and alleviate pain and nausea, improve quality of life, improve appetite and decrease stress in Crohn's patients (Martinez V. Iriondo De-Hond A, Borrelli F. Capasso R, Del Castillo M D, Abalo R. (2020) Cannabidiol and Other Non-Psychoactive Cannabinoids for Prevention and Treatment of Gastrointestinal Disorders: Useful Nutraceuticals?. Int J Mol Sci. 21(9):3067; Kienzl M, Storr M, Schicho R. (2020) Cannabinoids and Opioids in the Treatment of Inflammatory Bowel Diseases. Clin Transl Gastroenterol. 11(1):e00120; Perisetti A, Rimu A H, Khan S A, Bansal P, Goyal H. (2020) Role of cannabis in inflammatory bowel diseases. Ann Gastroenterol. 33(2):134-144).

Cannabinoids have been shown to decrease intestinal inflammation and reduce release of pro-inflammatory cytokines in animal models of colitis and intestinal in vitro models (Martinez V, Iriondo De-Hond A. Borrelli F, Capasso R. Del Castillo M D, Abalo R. (2020) Cannabidiol and Other Non-Psychoactive Cannabinoids for Prevention and Treatment of Gastrointestinal Disorders: Useful Nutraceuticals?. Int J Mol Sci. 21(9):3067; Hasenoehrl C, Taschler U, Storr M, Schicho R. (2016) The gastrointestinal tract—a central organ of cannabinoid signaling in health and disease. Neurogastroenterol Motil. 28(12):1765-1780; Borrelli F, Aviello G, Romano B, et al. (2009) Cannabidiol, a safe and non-psychotropic ingredient of the marijuana plant Cannabis sativa, is protective in a murine model of colitis. J Mol Med (Berl). 87(11):1111-1121; Jamontt J M, Molleman A, Pertwee R G, Parsons M E. (2010) The effects of Delta-tetrahydrocannabinol and cannabidiol alone and in combination on damage, inflammation and in vitro motility disturbances in rat colitis. Br J Pharmacol. 160(3):712-723).

For example, Pagano et al. recently demonstrated that cannabigerol (CBG) attenuated DNBS-induced intestinal inflammation and permeability, reduced myeloperoxidase (MPO) activity, Interleuken-1β (IL-1β) levels and was potentiated by omega-3 fatty acids (Pagano E, Iannotti F A, Piscitelli F, et al. (2021) Efficacy of combined therapy with fish oil and phytocannabinoids in murine intestinal inflammation. Phytother Res. 35(1):517-529). In observational studies, patients who use cannabinoids report relief of Crohn's symptoms such as decreased abdominal pain, decreased nausea, less diarrhea, and increased appetite, improved sleep quality, and less anxiety and fatigue (Ravikoff Allegretti J, Courtwright A, Lucci M. Korzenik J R, Levine J. (2013) Marijuana use patterns among patients with inflammatory bowel disease. Inflamm Bowel Dis. 19(13):2809-2814; Storr M, Devlin S, Kaplan G G, Panaccione R, Andrews C N. (2014) Cannabis use provides symptom relief in patients with inflammatory bowel disease but is associated with worse disease prognosis in patients with Crohn's disease. Inflamm Bowel Dis. 20(3):472-480; Hoffenberg E J, McWilliams S. Mikulich-Gilbertson S, Murphy B, Hoffenberg A, Hopfer C J (2019). Cannabis Oil Use by Adolescents and Young Adults With Inflammatory Bowel Disease. J Pediatr Gastroenterol Nutr. 68(3):348-352: Kerlin A M. Long M, Kappelman M. Martin C, Sandier R S. (2018) Profiles of Patients Who Use Marijuana for Inflammatory Bowel Disease. Dig Dis Sci. 63(6):1600-1604). A few small pilot clinical studies have been conducted with cannabis or THC cigarettes in Crohn's patients which showed improvement of daily activities, decreased pain, improved appetite, weight gain and decreased CDAI score, providing preliminary but inconclusive data on the effectiveness of cannabinoids as adjunctive treatment in Crohn's disease.

Accordingly, there is a need for new and improved cannabis therapeutics for treating inflammatory gastrointestinal disorders.

In various embodiments described herein, the present disclosure provides oral pulse-release dosage forms comprising one or more cannabinoid active pharmaceutical ingredients (API) and methods of using the oral pulse-release dosage forms described herein for treating inflammatory gastrointestinal disorders. In several embodiments, the oral pulse-release dosage forms described herein can target delivery the one or more cannabinoid active pharmaceutical ingredients (API) to one or more local regions of the gastrointestinal tract by timing the release of the one or more cannabinoid active pharmaceutical ingredients (API) following oral administration. In some embodiments, the oral pulse-release dosage forms described herein can comprise various combinations of pulse-release components that can include immediate release (IR) formulations and/or one or more delayed release (DR) formulations configured to release the one or more cannabinoid active pharmaceutical ingredients (API) as the pulse-release components transit the gastrointestinal tract following oral administration.

It is expected that the targeted delivery of the one or more cannabinoids locally to one or more inflamed regions of the gastrointestinal tract using the oral pulse-release dosage forms described herein can provide advantages for the treatment of inflammatory gastrointestinal disorders when compared to previous cannabinoid therapeutics. Such advantages may include but are not limited to, increased efficacy, lower dosing, decreased toxicity, decreased plasma bioavailability, decreased side-effects, such as decreased effects on systemic sites peripheral to the gastrointestinal tract, e.g. the central nervous system, among others, or any combination thereof. In some embodiments, the oral pulse-release dosage forms described herein may include a total dose of one or more cannabinoid API formulated in an immediate release formulation, one or more delayed-release formulations, or both. In some embodiments, following oral administration to a subject, at least a portion of the total dose of one or more cannabinoid API may be absorbed systemically into the plasma when released from an immediate release formulation. In some embodiments, at least a portion of the total dose of one or more cannabinoid API may have decreased systemic absorption, decreased plasma bioavailability, or both, when released from a delayed release formulation. The oral pulse-release dosage forms described herein can provide timed release of the one or more cannabinoid API throughout the day, Accordingly, the oral pulse-release dosage forms described herein can provide advantages over other cannabinoid therapeutics that may require two, three or four times a day administration of the one or more cannabinoid API.

### A. Active Pharmaceutical Ingredients (API's)

In some embodiments, an API of the present disclosure can be a cannabinoid. The term “cannabinoid” as used herein refers to several classes of compounds that can be found in plants of the genus Cannabis. There are at least 144 different cannabinoids that have been isolated from cannabis, exhibiting varied effects. Synthetic cannabinoids encompass a variety of distinct chemical classes: the classical cannabinoids structurally related to THC, the nonclassical cannabinoids (cannabimimetics) including the aminoalkylindoles, 1,5-diarylpyrazoles, quinolines, and arylsulfonamides as well as eicosanoids related to endocannabinoids.

The classical cannabinoids are concentrated in a viscous resin of cannabis plants produced in structures known as glandular trichomes. The main classes of cannabinoids include, without limitation, THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol), CBV (cannabivarin), THCC (tetrahydrocannabiorcol), THCV (tetrahydrocannabivarin), THCP (tetrahydrocannabiphorol), CBDV (cannabidivarin), CBCV (cannabichromevarin), CBGV (cannabigerovarin), CBGM (cannabigerol monomethyl ether), CBE (cannabielsoin), and CBT (cannabicitran). Cannabinoids of the present disclosure include, without limitation: Cannabigerol-type (CBG) cannabinoids, such as Cannabigerol monomethyl ether, Cannabinerolic acid A, Cannabigerovarin, Cannabigerolic acid A, Cannabigerolic acid A monomethyl ether, and Cannabigerovarinic acid A; Cannabichromene-type (CBC) cannabinoids, such as (±)-Cannabichromene, (±)-Cannabichromenic acid A, (±)-Cannabivarichromene, (±)-Cannabichromevarin, and (±)-Cannabichromevarinic acid A; Cannabidiol-type (CBD) cannabinoids, such as (−)-Cannabidiol, Cannabidiol momomethyl ether, Cannabidiol-C₄, (−)-Cannabidivarin, Cannabidiorcol, Cannabidiolic acid, and Cannabidivarinic acid; Cannabinodiol-type (CBND) cannabinoids, such as Cannabinodiol and Cannabinodivarin; Tetrahydrocannabinol-type (THC) cannabinoids, such as Δ⁹-Tetrahydrocannabinol, Δ⁹-Tetrahydrocannabinol-C₄, Δ⁹-Tetrahydrocannabivarin, Δ⁹-Tetrahydrocannabiorcol, Δ⁹-Tetrahydro-cannabinolic acid A, Δ⁹-Tetrahydro-cannabinolic acid B, Δ⁹-Tetrahydro-cannabinolic acid-C₄ A and/or B, Δ⁹-Tetrahydro-cannabivarinic acid A, Δ⁹-Tetrahydro-cannabiorcolic acid A and/or B, (−)-Δ⁸-trans-(6aR,10aR)-Δ⁸-Tetrahydrocannabinol, (−)-Δ⁸-trans-(6aR,10aR)-Tetrahydrocannabinolic acid A, and (−)-(6aS,10aR)-Δ⁹-Tetrahydrocannabinol; Cannabinol-type (CBN) cannabinoids, such as Cannabinol, Cannabinol-C₄, Cannabivarin, Cannabinol-C₂, Cannabiorcol, Cannabinolic acid A, and Cannabinol methyl ether; Cannabitriol-type (CBT) cannabinoids, such as (−)-(9R,10R)-trans-Cannabitriol, (+)-(9S,10S)-Cannabitriol, (±)-(9R,10S/9S,10R)-Cannabitriol, (−)-(9R,10R)-trans-10-O-Ethyl-cannabitriol, (±)-(9R,10R/9S,10S)-Cannabitriol-C₃, 8,9-Dihydroxy-Δ^{6a(10a)}-tetrahydrocannabinol, Cannabidiolic acid A cannabitriol ester, (−)-(6aR,9S,10S,10aR)-9,10-Dihydroxy-hexahydrocannabinol, Cannabiripsol, (−)-6a,7,10a-Trihydroxy-Δ⁹-tetrahydrocannabinol, 10-Oxo-^{Δ6a(10a)}-tetrahydrocannabinol; Cannabielsoin-type (CBE) cannabinoids such as (5aS,6S,9R,9aR)-Cannabielsoin, (5aS,6S,9R,9aR)-C₃-Cannabielsoin, (5aS,6S,9R,9aR)-Cannabielsoic acid A, (5aS,6S,9R,9aR)-Cannabielsoic acid B, (5aS,6S,9R,9aR)-C₃-Cannabielsoic acid B, Cannabiglendol-C₃, Dehydrocannabifuran, and Cannabifuran; Isocannabinoids such as (−)-Δ⁷-trans-(1R,3R,6R)-Isotetrahydrocannabinol, (±)-Δ⁷-1,2-cis-(1R,3R,6S/1S,3S,6R)-Isotetrahydro-cannabivarin, and (−)-Δ⁷-trans-(1R,3R,6R)-Isotetrahydrocannabivarin; Cannabicyclol-type (CBL) cannabinoids such as (±)-(1aS,3aR,8bR,8cR)-Cannabicyclol, (±)-(1aS,3aR,8bR,8cR)-Cannabicyclolic acid A, and (±)-(1aS,3aR,8bR,8cR)-Cannabicyclovarin; Cannabicitran-type (CBT) cannabinoids such as Cannabicitran; and Cannabichromanone-type (CBCN) cannabinoids such as Cannabichromanone, Cannabichromanone-C₃, and Cannabicoumaronone.

In some embodiments, the term “cannabinoid” as used herein refers to compounds that may be extracted from, and/or derived from Cannabis plants using methods known in the art. For example, cannabinoids can be separated from the cannabis plant by extraction with organic solvents or using supercritical solvent extraction with carbon dioxide. Cannabinoids may be obtained in oils extracted from cannabis plants, and thereafter further isolated, purified, and converted into derivatives. In some embodiments, the term “cannabinoid” as used herein may also refer to such compounds that may be produced synthetically or using recombinant biotechnology methods, as may be identified by persons of ordinary skill in the art.

Cannabinoids of the present disclosure may include stereoisomers thereof, and modified forms thereof, such as a pharmaceutically acceptable salt, ester, derivative, analog, prodrug, hydrate, or solvate thereof.

In some embodiments, a pharmaceutical compositions of the present disclosure can include, without limitation, one or more cannabinoids, two or more cannabinoids, or three or more cannabinoids. In some embodiments, the cannabinoid may be CBG, CBD, THC, or any combinations thereof.

Cannabigerol (CBG) refers to the decarboxylated form of cannabigerolic acid, the parent molecule from which other cannabinoids are synthesized. Cannabigerol is a minor constituent of cannabis. During plant growth, most of the cannabigerol is converted into other cannabinoids, primarily tetrahydrocannabinol (THC) or cannabidiol (CBD), leaving about 1% cannabigerol in the plant. In the cannabis plant, the biosynthesis of cannabigerol begins by loading hexanoyl-CoA onto a polyketide synthase assembly protein and subsequent condensation with three molecules of malonyl-CoA. This polyketide is cyclized to olivetolic acid via olivetolic acid cyclase, and then prenylated with a ten carbon isoprenoid precursor, geranyl pyrophosphate, using an aromatic prenyltransferase enzyme, geranyl-pyrophosphate-olivetolic acid geranyltransferase, to biosynthesize cannabigerolic acid, which can then be decarboxylated to yield cannabigerol. Contrary to the major psychoactive cannabinoid THC, cannabigerol antagonizes CB1 receptors and is both an α₂-adrenergic receptor agonist and moderate 5-HT_{1A} receptor antagonist. Cannabigerol displays CB1 and CB2 binding affinity.

The chemical structure of CBG may be represented as Formula I:

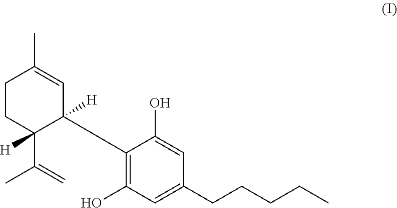 [SMILES: C=C(C)[C@@H]1CCC(C)=C[C@H]1c1c(O)cc(CCCCC)cc1O]

The best studied cannabinoids include tetrahydrocannabinol (THC) and cannabidiol (CBD). Δ⁹-tetrahydrocannabinol (Δ⁹-THC or THC), is also known by its International Non-Proprietary Name (INN) as dronabinol. The unsaturated bond in the cyclohexene ring is located between C-9 and C-10 in the more common dibenzopyran ring numbering system. There are four stereoisomers of THC, but only the (−)-trans isomer occurs naturally (CAS-1972-08-03). The fully systematic name for this THC isomer is (−)-(6aR,10aR)-6,6,9-trimethyl-3-pentyl-6a,7,8,10a-tetrahydro-6H-benzo[c]chromen-1-ol. Two related substances, Δ⁹-tetrahydrocannabinol-2-oic acid and Δ⁹-tetrahydrocannabinol-4-oic acid (THCA), are also present in cannabis, sometimes in large amounts. During smoking, THCA is partly converted to THC. The active isomer Δ⁸-THC, in which the unsaturated bond in the cyclohexene ring is located between C-8 and C-9, is found in much smaller amounts. Other closely related substances that occur in cannabis include cannabidiol (CBD) which has quite different pharmacological effects to THC.

THC is the most known active component of cannabis. The pharmacology of cannabis is complicated by the presence of a wide range of cannabinoids. At small doses, cannabis produces euphoria, relief of anxiety, sedation and drowsiness. In some respects, the effects are similar to those caused by alcohol. Anandamide has been identified as the endogenous ligand for the cannabinoid receptor and has pharmacological properties similar to those of THC. When cannabis is smoked, THC can be detected in plasma within seconds of inhalation; it has a half-life of 2 hours. Following smoking of the equivalent of 10-15 mg over a period of 5-7 minutes, peak plasma levels of Δ⁹-THC are around 100 μg/L. It is highly lipophilic and widely distributed in the body. Two active metabolites are formed: 11-hydroxy-Δ⁹-THC and 8p-hydroxy-Δ⁹-THC. The first is further metabolized to Δ⁹-THC-11-oic acid. Two inactive substances are also formed—8α-hydroxy-Δ⁹-THC and 8α,11-dihydroxy-Δ⁹-THC—and many other minor metabolites, most of which appear in the urine and feces as glucuronide conjugates. Some metabolites can be detected in the urine for up to 2 weeks following smoking or ingestion.

Bioavailability following the smoking route was reported as 2-56%, due in part to intra- and inter-subject variability in smoking dynamics, which contributes to uncertainty in dose delivery. The number, duration, and spacing of puffs, hold time, and inhalation volume, or smoking topography, greatly influences the degree of drug exposure (Huestis M., “Human Cannabinoid Pharmacokinetics” Chem Biodivers. 2007 August; 4(8): 1770-1804).

The chemical structure of Δ⁹-THC may be represented as Formula II:

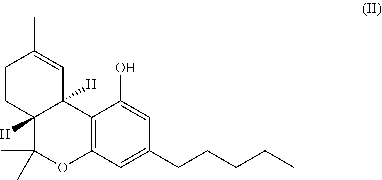 [SMILES: CCCCCc1cc(O)c2c(c1)OC(C)(C)[C@@H]1CCC(C)=C[C@@H]21]

The chemical structure of CBD may be represented as Formula II:

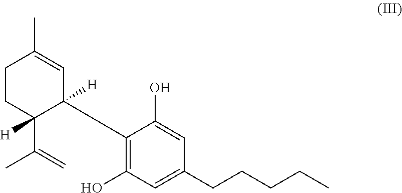 [SMILES: C=C(C)[C@@H]1CCC(C)=C[C@H]1c1c(O)cc(CCCCC)cc1O]

In the cannabis plant, CBD-carboxylic acid is produced through the same metabolic pathway as THC, until the penultimate step, where CBDA synthase performs catalysis instead of THCA synthase. Cannabidiol has low affinity for the cannabinoid CB₁ and CB₂ receptors, although it can act as an antagonist of CB₁/CB₂ agonists despite this low affinity. The oral bioavailability of cannabidiol is approximately 6% in humans, while its bioavailability via inhalation is approximately 31%. Cannabidiol may be an antagonist of GPR55, a G protein-coupled receptor and putative cannabinoid receptor that is expressed in the caudate nucleus and putamen in the brain. It also may act as an inverse agonist of GPR3, GPR6, and GPR12. CBD has been shown to act as a serotonin 5-HT_{1A} receptor partial agonist. At higher concentrations, CBD acts as an inverse agonist of 5-HT1A receptors. CBD is also an allosteric modulator of the μ- and δ-opioid receptors as well. The pharmacological effects of CBD may involve PPARγ agonism, inhibition of voltage-gated cation channels, and intracellular calcium release.

The pharmaceutical formulations described herein are provided to meet the need in the art for oral dosage forms that provide controlled and targeted release into particular regions of the gastrointestinal tract of a subject, in order to provide delivery of cannabinoid APIs described herein that in several embodiments may be useful for treating inflammatory gastrointestinal disorders.

### B. Pharmaceutical Formulations

In some embodiments, the present disclosure provides oral pulse-release compositions and dosage forms thereof, comprising a total dose, e.g. a total daily dose, of one or more cannabinoid active pharmaceutical ingredients.

The terms “pulse-release” or “pulsatile” formulation, as used herein refers in general to release of a portion of a total API dose, in a burst or as a sustained release, followed by periods of little or no release (e.g., a lag phase or a decrease in release rate) in a defined temporal pattern. In particular, oral pulsatile drug release pertains to the burst delivery or sustained delivery of drugs following a temporal pattern from the time of oral administration. In some embodiments, the pulse-release formulations of the present disclosure may combine a range of formulation approaches, including single- or multiple-unit immediate-release, delayed-release, and/or sustained-release components. For example, in some embodiments, the delayed release components in the pulsatile formulations described herein may be configured to release an API at a desired site within the gastrointestinal tract, and/or release of the API after a defined time period. In some embodiments, an oral pulse-release dosage form of the present disclosure may comprise one or more immediate release oral pharmaceutical formulation components, one or more delayed release oral pharmaceutical formulation components, one or more sustained release oral pharmaceutical formulation components, or any combinations thereof.

In some embodiments, when administered orally to a subject, an oral pulse-release dosage form of the present disclosure is configured to provide release of a dosage, e.g. a daily dosage, of the one or more cannabinoids into the gastrointestinal tract of the subject, divided into at least 2 separate releases, or pulses separated by at least 1 hour, and not more than 6 hours, between releases. In some embodiments, when administered orally to a subject, an oral pulse-release dosage form of the present disclosure is configured to provide release of a total dosage, e.g. a total daily dosage, of the one or more cannabinoids into the gastrointestinal tract of the subject, divided into at least 3, 4, or 5 separate releases, or pulses separated by at least 1 hour, and not more than 6 hours, between releases.

In some embodiments, an oral pulse-release dosage form of the present disclosure may provide a total dose, e.g. a total daily dose of a cannabinoid API. In some embodiments, an oral pulse-release dosage form of the present disclosure may provide a partial dose, e.g. a partial daily dose, of a cannabinoid API. In some embodiment, a plurality, e.g. 2, 3, or more of the oral pulse-release dosage form of the present disclosure may be administered to a subject to provide a total dose, e.g. a total daily dose. In some embodiments, the oral pulse-release dosage form of the present disclosure may be administered once, twice, three times or more, per day to provide a total daily dose of a cannabinoid API.

In some embodiments, the pulse-release dosage forms of the present disclosure are configured to release a portion of an API in the upper gastrointestinal tract, e.g. into the gastric region (e.g. in the stomach), and the remaining portion in the lower gastrointestinal tract, e.g., into the small intestine e.g. distal duodenum, or colon, or other distal sites in the gastrointestinal tract.

Accordingly, in some embodiments, the present disclosure provides an oral pulse-release dosage form comprising a total dose of one or more cannabinoid active pharmaceutical ingredients (API), wherein the oral pulse-release dosage form comprises a first pulse-release component (C₁) comprising a first portion (P₁) of the one or more cannabinoid API (API-P₁), and a second pulse-release component (C₂) comprising a second portion (P₂) of the one or more cannabinoid API (API-P₂). The total dose of each of the one or more cannabinoid API may be divided between the first portion (P₁) in the first pulse-release component (C₁) and the second portion (P₂) in the at least second pulse-release component (C₂). When the pulse-release dosage form is placed in a simulated gastrointestinal environment, the pulse-release dosage form provides release of the one or more cannabinoid API-P₂ beginning from 1 to 6 hours after release of the API-P₁ begins.

The term “simulated gastrointestinal environment” is intended to be used herein to convey its ordinary meaning as understood in the art, and is understood in a broad sense to mean conditions that mimic oral administration, for example, an aqueous medium solution, or series of aqueous media solutions, into which a pharmaceutical composition may be added to study it's release profile, such as an aqueous environment of low pH, 1-5 for example, followed after a period of up to about 2 hours with immersion in a higher pH aqueous environment, such as pH 6.8, for example, or a 3 stage environment in which the low pH is followed by an intermediate pH of about 6 wherein the environments are maintained at about 37.0° C. In some embodiments a simulated gastrointestinal environment may be, for example, an aqueous solution of 0.1N HCl for two hours, followed by phosphate buffered solution at pH 6.0 and 37° C.±0.5° C. for four hours, followed by phosphate buffered solution at pH 7.2 and 37° C.±0.5° C. for up to fourteen hours, and sampling of the media of the simulated gastrointestinal environment at appropriate time intervals, e.g. at 1, 1.5, 2, 3, 3.5, 4, 5, 6, 7, 8, 9, 10, 11, 12, 13, 14, 15, 16, 17, 18, 19 and 20 hours.

For example, a two-stage dissolution test is described in USP General Chapter <711> Dissolution, in which the integrity of an enteric coating is determined in an acidic environment and the drug release is measured in a neutral environment. The test can be performed using either medium-addition or medium exchange methods; both may start with an acid stage in 0.1 N hydrochloric acid for two hours and follow with a buffer stage in phosphate buffer e.g. at pH 6.8 or pH 7.2 for specific times as needed for the individual drug product. Medium addition or medium exchange procedures may be used. For the medium-addition approach, a designated amount of concentrated phosphate buffer may be added to the dissolution vessel to neutralize the medium to the target pH before the buffer stage starts. The operations of adding the buffer and adjusting the pH may be completed within 5 min. For the medium-exchange approach, the acid medium may be drained after two hours, and a full amount of pH 6.8 or pH 7.2 buffer may be added to the same vessel for the buffer stage. The dosage unit is typically left undisturbed during the medium change. Alternatively, the vessel containing the acid can be removed and replaced with another vessel containing the buffer, and the dosage unit transferred to the new vessel.

Parameters of suitable API-specific biorelevant dissolution methods such as in vitro dissolution analysis can be determined by skilled persons without undue experimentation upon reading the present disclosure. For example, and without limitation, an in-vitro non-sink, gastric transfer dissolution method may be used to analyze the release and dissolution of API.

At pre-determined time points, samples may be drawn from the dissolution vessel and centrifuged using an ultracentrifuge. The supernatants may be further diluted using an HPLC diluent and analyzed by a suitable HPLC method. Parameters such as peak release rate (PRR) and the area under drug dissolution curve (AUDC), among others, may be calculated, e.g. by the linear trapezoidal method.

Each of the pulse-release components that may be combined to provide the oral pulse-release dosage form may be analyzed separately by in vitro dissolution tests, to determine the pulse-release timing for each pulse-release component of the oral pulse-release dosage form. The “beginning” of release from a pulse-release component may be defined as the time at which a detectable amount of the API is present in the dissolution media, or when a certain percentage of the total dose is detected in the dissolution media, e.g. at least 1%, 5%, or 10% of the total amount of the API present in the particular pulse-release component.

Accordingly, in some embodiments, the present disclosure provides an oral pulse-release dosage form which is comprised of at least two pulse-release components. Such dosage forms are formulated so that each of the pulse-release components releases the one or more cannabinoid API with a different temporal release profile.

In some embodiments, the present disclosure provides an oral pulse-release dosage form which is comprised of two, or up to three, four or five pulse-release components formulated so that each of the pulse-release components in the oral pulse-release dosage form releases the one or more cannabinoid API with a different temporal release profile.

Accordingly, in some embodiments, the oral pulse-release dosage form further comprises a third pulse-release component (C₃) comprising a third portion (P₃) of the one or more cannabinoid API (API-P₃), wherein the total dose of each of the one or more cannabinoid API is further divided between the first portion (P₁) in the first pulse-release component (C₁), the second portion (P₂) in the second pulse-release component (C₂), and the third portion (P₃) in the third pulse-release component (C₃). Accordingly, in some embodiments, when the pulse-release dosage form is placed in a simulated gastrointestinal environment, (e.g., an aqueous solution of 0.1N HCl for two hours, followed by phosphate buffered solution at pH 6.0 and 37° C.±0.5° C. for four hours, followed by phosphate buffered solution at pH 7.2 and 37° C.±0.5° C. for up to fourteen hours), the pulse-release dosage form provides release of the one or more cannabinoid API-P₃ beginning from 1 to 6 hours after release of the API₁P₂ begins.

In some embodiments, the oral pulse-release dosage form further comprises a fourth pulse-release component (C₄) comprising a fourth portion (P₄) of the one or more cannabinoid API (API-P₄), wherein the total dose of each of the one or more cannabinoid API is further divided between the first portion (P₁) in the first pulse-release component (C₁), the second portion (P₂) in the second pulse-release component (C₂), the third portion (P₃) in the third pulse-release component (C₃), and the fourth portion (P₄) in the fourth pulse-release component (C₄); Accordingly, in some embodiments, when the pulse-release dosage form is placed in a simulated gastrointestinal environment (e.g., an aqueous solution of 0.1N HCl for two hours, followed by phosphate buffered solution at pH 6.0 and 37° C.±0.5° C. for four hours, followed by phosphate buffered solution at pH 7.2 and 37° C.±0.5° C. for up to fourteen hours), the pulse-release dosage form provides release of the one or more cannabinoid API-P₄ beginning from 1 to 6 hours after release of the API-P₃ begins.

In some embodiments, the oral pulse-release dosage form further comprises a fifth pulse-release component (C₅) comprising a fifth portion (P₅) of the one or more cannabinoid API (API-P₅), wherein the total dose of each of the one or more cannabinoid API is further divided between the first portion (P₁) in the first pulse-release component (C₁), the second portion (P₂) in the second pulse-release component (C₂), the third portion (P₃) in the third pulse-release component (C₃), the fourth portion (P₄) in the fourth pulse-release component (C₄), and the fifth portion (P₅) in the fifth pulse-release component (C₅). Accordingly, in some embodiments, when the pulse-release dosage form is placed in a simulated gastrointestinal environment (e.g., an aqueous solution of 0.1N HCl for two hours, followed by phosphate buffered solution at pH 6.0 and 37° C.±0.5° C. for four hours, followed by phosphate buffered solution at pH 7.2 and 37° C.±0.5° C. for up to fourteen hours), the pulse-release dosage form provides release of the one or more cannabinoid API-P₅ beginning from 1 to 6 hours after release of the API-P₄ begins.

Analysis of the cannabinoid API concentration present in the media of the simulated gastric environment at various timepoints can provide data on the rate of release of the cannabinoid API into the media at various time points. For example, an oral pulse-release dosage form may provide a highest or peak rate of release (e.g. in nanograms released into the media per hour) between certain sampling timepoints. Such a time of peak release rate (PRR) observed in the simulated gastric environment may therefore provide a proxy for the time of peak release rate of a cannabinoid API into the gastrointestinal tract of a subject. Because the oral pulse-release dosage forms described herein are configured to release a total dose of a cannabinoid API in two or more pulses, two or more PRR may be observed from the samples at various timepoints from the media of the simulated gastrointestinal environment, each PRR separated by a lower, or trough, release rate. Accordingly, when a pulse-release dosage form of the present disclosure is placed in a simulated gastrointestinal environment, the pulse-release dosage form may provide a first PRR, followed by a second PRR, a third PRR, a fourth PRR, and up to a fifth PRR.

In some embodiments, when the pulse-release dosage form is placed in a simulated gastrointestinal environment, the pulse-release dosage form may provide a second time of peak release rate (PRR₂) of each of the one or more cannabinoid API (PRR₂API) from about 1 to 6 hours after a first time of peak release rate (PRR₁) of each of the one or more cannabinoid API (PRR₁API). In some embodiments, when the pulse-release dosage form is placed in a simulated gastrointestinal environment, a first time of PRR (PRR₁) of each of the one or more cannabinoid API may be after 1, 2, 3, 4, 5, 6, 7, or 8 hours, and a second time of PRR (PRR₂) may be from about 1 to 6 hours after the first time of PRR (PRR₁). Accordingly, as described herein, the first pulse-release component (C₁) may comprise an immediate-release (IR) formulation of the one or more cannabinoid API, or a delayed-release (DR) formulation of the one or more cannabinoid API.

In some embodiments, when the oral pulse-release dosage form further comprises a third pulse-release component (C₃), the oral pulse-release dosage form may provide a third time of peak release rate (PRR₃) of each of the one or more cannabinoid API (PRR₃API) from about 1 to 6 hours after the second time of peak release rate (PRR₂) of each of the one or more cannabinoid API (PRR₂API).

In some embodiments, when the oral pulse-release dosage form further comprises a fourth pulse-release component (C₄), the oral pulse-release dosage form may provide a fourth time of peak release rate (PRR₄) of each of the one or more cannabinoid API (PRR₄API) from about 1 to 6 hours after the third time of peak release rate (PRR₃) of each of the one or more cannabinoid API (PRR₃API).

In some embodiments, when the oral pulse-release dosage form further comprises a fifth pulse-release component (C₅), the oral pulse-release dosage form may provide a fifth time of peak release rate (PRR₅) of each of the one or more cannabinoid API (PRR₅API) from about 1 to 6 hours after the fourth time of peak release rate (PRR₄) of each of the one or more cannabinoid API (PRR₄API).

In some embodiments, the PRR₁ can be after about 1, 2, 3, 4, 5, 6, 7, 8, 9, 10, 11, 12, 13, 14, 15, or 16 hours. In some embodiments, the PRR₂ can be after about 2, 3, 4, 5, 6, 7, 8, 9, 10, 11, 12, 13 14, 15, 16, or 17 hours. In some embodiments, the PRR₃ can be after about 3, 4, 5, 6, 7, 8, 9, 10, 11, 12, 13 14, 15, 16, 17, or 18 hours. In some embodiments, the PRR₄ can be after about 4, 5, 6, 7, 8, 9, 10, 11, 12, 13 14, 15, 16, 17, 18, or 19 hours. In some embodiments, the PRR₅ can be after about 5, 6, 7, 8, 9, 10, 11, 12, 13 14, 15, 16, 17, 18, 19, or 20 hours.

Accordingly, it is an aspect of the compositions and methods of the present disclosure that the disclosed oral pulse-release dosage forms provide novel release profiles in vitro and in vivo when orally administered to a subject. In some embodiments, following oral administration to a subject of an oral pulse-release dosage form described herein, two or more maximum serum concentrations (Cₘₐₓ), e.g., a first Cₘₐₓ (Cₘₐₓ1) and second Cₘₐₓ (Cₘₐₓ2), and up to a third Cₘₐₓ (Cₘₐₓ3), a fourth Cₘₐₓ (Cₘₐₓ4), or a fifth Cₘₐₓ (Cₘₐₓ5) of the one or more cannabinoid API may be observed at various timepoints in the serum of the subject. Each Cₘₐₓ may be separated by a lower, or trough, serum concentration of the one or more cannabinoid API. Accordingly, in some embodiments, when a pulse-release dosage form of the present disclosure is administered to a subject, the pulse-release dosage form may provide a Cₘₐₓ1 followed by a Cₘₐₓ2, and may be followed by up to a Cₘₐₓ3, which may be followed by a Cₘₐₓ4, which may be followed by a Cₘₐₓ5.

In some embodiments, the Cₘₐₓ1 can be about 1, 2, 3, 4, 5, 6, 7, 8, 9, 10, 11, 12, 13, 14, 15, or 16 hours after administration. In some embodiments, the Cₘₐₓ2 can be about 2, 3, 4, 5, 6, 7, 8, 9, 10, 11, 12, 13 14, 15, 16, or 17 hours after administration. In some embodiments, the Cₘₐₓ3 can be about 3, 4, 5, 6, 7, 8, 9, 10, 11, 12, 13 14, 15, 16, 17, or 18 hours after administration. In some embodiments, the Cₘₐₓ4 can be about 4, 5, 6, 7, 8, 9, 10, 11, 12, 13 14, 15, 16, 17, 18, or 19 hours after administration. In some embodiments, the Cₘₐₓ5 can be about 5, 6, 7, 8, 9, 10, 11, 12, 13 14, 15, 16, 17, 18, 19, or 20 hours after administration.

It is an aspect of the compositions and methods of the present disclosure that the delayed release (DR) formulations described herein can provide a lag time of from 1, 2, 3, 4, 5, 6, 7, 8, 9, 10, 11, or 12 hours followed by a period that includes release of the one or more cannabinoid API in an ascending rate. It is a further aspect of the compositions and methods of the present disclosure that, when a pulse-release component is a delayed release (DR) formulation, the one or more cannabinoid API can begin to be slowly released during the lag time. This release is determined by the composition of the delayed-release (DR) formulations as described herein. Some examples of a small release during the lag time are those in which no more than about 10% of the one or more cannabinoid API is released during a 1, 2, 3, 4, 5, 6, 7, 8, 9, 10, 11 or 12-hour lag. It is also understood that, in some embodiments, a smaller percentage, such as less than 10%, 9%, 8%, 7%, 6%, 5%, 4%, 3%, 2% or 1%, can be released during the first 1, 2, 3, 4, 5, 6, 7, 8, 9, 10, 11, or 12 hours after oral administration.

In some embodiments, the oral pulse-release dosage form may comprise a total dose, e.g. a total daily dose, of a cannabinoid API from 1 mg to 300 mg, for example, about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300 mg.

In some embodiments, the cannabinoid may comprise CBG. In some embodiments, the oral pulse-release dosage form may comprise a total dose of CBG of up to about 10, 20, 30, 40, 50, 60, 70, 80, 90, 100, 110, 120, 130 140, 150, 160, 170, 180, 190, 200, 210, 220, 230, 240, 250, 260, 270, 280, 290, or 300 mg.

In some embodiments, the oral pulse-release dosage form may comprise CBG and no other cannabinoids. In some embodiments, the oral pulse-release dosage form may comprise CBG and CBD. In some embodiments, the oral pulse-release dosage form may comprise a total dose of CBD of up to 5, 10, 15, 20, 25, 30, 35, 40, 45, 50, 55, 60, 65, 70, 75, or 80 mg.

In some embodiments, the oral pulse-release dosage form may comprise CBG, CBD, and THC. In some embodiments, the oral pulse-release dosage form may comprise a total dose of THC of up to about 0.2, 0.4, 0.6, 0.8, 1.0, 1.2, 1.4, 1.6, 1.8, 2.0, 2.2, 2.4, 2.6, 2.8, 3.0, 3.2, 3.4, 3.6, 3.8, or 4.0 mg.

In some embodiments, the total dose of THC may be up to 2 mg to decrease hallucinogenic effects. In some embodiments, the total dose of CBD may be up to 40 mg to decrease the CNS effects. In some embodiments, up to 30% of the total dose of CBG may be comprised in an immediate release (IR) formulation to decrease systemic absorption of CBG from the oral pulse-release dosage form. In some embodiments, at least 70%, 71%, 72%, 73%, 74%, 75%, 76%, 77%, 78%, 79%, 80%, 81%, 82%, 83%, 84%, 85% 86%, 87%, 88%, 89%, 90%, 91%, 92%, 93%, 94%, 95%, 96%, 97%, 98%, 99% or 100% of the total dose of CBG in the oral pulse-release dosage form may be comprised in a delayed release (DR) formulation to decrease the systemic absorption of CBG.

In some embodiments, release of the total dose of the one or more cannabinoid API from the oral pulse-release dosage form may occur in up to 20 hours, up to 19 hours, up to 18 hours, up to 17 hours, up to 16 hours, up to 15 hours, up to 14 hours, up to 13 hours, up to 12 hours, up to 11 hours, up to 10 hours, up to 9 hours, up to 8 hours, or less.

In some embodiments, the first pulse-release component (C₁) can be an immediate release (IR) formulation or a delayed-release (DR) formulation. In some embodiments, the first pulse-release component (C₁) comprises from 10% to 50% (e.g. about 10%, 20%, 30%, 40% or 50%) of the total dose independently of each of the one or more cannabinoid API. In some embodiments, the second pulse-release component (C₂) is a delayed-release (DR) formulation comprising from 50% to 90% (e.g. about 50%, 60%, 70%, 80%, or 90%) of the total dose independently of each of the one or more cannabinoid API.

In some embodiments, the first pulse-release component (C₁) can be an immediate release (IR) formulation or a delayed-release (DR) formulation. In some embodiments, the first pulse-release component (C₁) may comprise from 10% to 30% (e.g. about 10%, 20%, or 30%) of the total dose independently of each of the one or more cannabinoid API. In some embodiments, the second pulse-release component (C₂) and the third pulse-release component (C₃) can be delayed-release (DR) formulations. In some embodiments, the second pulse-release component (C₂) may comprise from 20% to 40% (e.g. about 20%, 30% or 40%) of the total dose independently of each of the one or more cannabinoid API. In some embodiments, the third pulse-release component (C₃) may comprise from 30% to 70% (e.g. about 30%, 40%, 50%, 60% or 70%) of the total dose independently of each of the one or more cannabinoid API. In some embodiments, together the second pulse-release component (C₂) and the third pulse-release component (C₃) may comprise from 70% to 90% of the total dose independently of each of the one or more cannabinoid API.

In some embodiments, the first pulse-release component (C₁) can be an immediate release (IR) formulation or a delayed-release (DR) formulation, wherein the first pulse-release component (C₁) comprises from 5% to 20% (e.g. about 5%, 10%, 15% or 20%) of the total dose independently of each of the one or more cannabinoid API. In some embodiments, the second pulse-release component (C₂), the third pulse-release component (C₃), and the fourth pulse-release component (C₄) can be delayed-release (DR) formulations. In some embodiments, the second pulse-release component (C₂) can comprise from 15% to 30% (e.g. about 15%, 20%, 25% or 30%) of the total dose independently of each of the one or more cannabinoid API. In some embodiments, the third pulse-release component (C₃) can comprise from 20% to 40% (e.g. about 20%, 25%, 30%, 35% or 40%) of the total dose independently of each of the one or more cannabinoid API. In some embodiments, the fourth pulse-release component (C₄) can comprise from 10% to 60% (e.g. about 10%, 20%, 30%, 40%, 50% or 60%) of the total dose independently of each of the one or more cannabinoid API. In some embodiments, together the second pulse-release component (C₂), the third pulse-release component (C₃), and the fourth pulse-release component (C₄) can comprise from 80% to 95% (e.g. about 80%, 85%, 90% or 95%) of the total dose independently of each of the one or more cannabinoid API.

In some embodiments, the first pulse-release component (C₁) can be an immediate release (IR) formulation or a delayed-release (DR) formulation, wherein the first pulse-release component (C₁) comprises from 5% to 15% (e.g. about 5%, 10% or 15%) of the total dose independently of each of the one or more cannabinoid API. In some embodiments, the second pulse-release component (C₂), the third pulse-release component (C₃), the fourth pulse-release component (C₄), and the fifth pulse-release component (C₅) can be delayed-release (DR) formulations. In some embodiments, the second pulse-release component (C₂), can comprise from 10% to 20% (e.g. about 10%, 15% or 20%) of the total dose independently of each of the one or more cannabinoid API. In some embodiments, the third pulse-release component (C₃) can comprise from 15% to 25% (e.g. about 15%, 20% or 25%) of the total dose independently of each of the one or more cannabinoid API. In some embodiments, the fourth pulse-release component (C₄) can comprise from 15% to 25% (e.g. about 15%, 20% or 25%) of the total dose independently of each of the one or more cannabinoid API. In some embodiments, the fifth pulse-release component (C₅) can comprise from 15% to 55% (e.g. about 15%, 20%, 25%, 30%, 35%, 40%, 45%, 50% or 55%) of the total dose independently of each of the one or more cannabinoid API. In some embodiments, together the second pulse-release component (C₂), the third pulse-release component (C₃), the fourth pulse-release component (C₄) and the fifth pulse-release component (C₅) comprise from 85% to 95% (e.g. about 85%, 90%, or 95%) of the total dose independently of each of the one or more cannabinoid API.

In some embodiments, the first pulse-release component (C₁) may comprise up to 10%, up to 20%, up to 30%, up to 40%, up to 50%, up to 60%, up to 70%, up to 80%, up to 90%, or up to 100% of the total dose independently of each of the one or more cannabinoid API.

In some embodiments, the second pulse-release component (C₂) may comprise up to 10%, up to 20%, up to 30%, up to 40%, up to 50%, up to 60%, up to 70%, up to 80%, up to 90%, or up to 100% of the total dose independently of each of the one or more cannabinoid API.

In some embodiments, the third pulse-release component (C₃) may comprise up to 10%, up to 20%, up to 30%, up to 40%, up to 50%, up to 60%, up to 70%, up to 80%, up to 90%, or up to 100% of the total dose independently of each of the one or more cannabinoid API.

In some embodiments, the fourth pulse-release component (C₄) may comprise up to 10%, up to 20%, up to 30%, up to 40%, up to 50%, up to 60%, up to 70%, up to 80%, up to 90%, or up to 100% of the total dose independently of each of the one or more cannabinoid API.

In some embodiments, the fifth pulse-release component (C₅) may comprise up to 10%, up to 20%, up to 30%, up to 40%, up to 50%, up to 60%, up to 70%, up to 80%, up to 90%, or up to 100% of the total dose independently of each of the one or more cannabinoid API.

In some embodiments, the first pulse-release component (C₁) may comprise up to 90%, up to 91%, up to 92%, up to 93%, up to 94%, up to 95%, up to 96%, up to 97%, up to 98%, up to 99%, or up to 100% of the total THC dose. In some embodiments, the third pulse-release component (C₃) may comprise up to 90%, up to 91%, up to 92%, up to 93%, up to 94%, up to 95%, up to 96%, up to 97%, up to 98%, up to 99%, or up to 100% of the total CBG dose. In some embodiments, the second pulse-release component (C₂) may comprise up to 60%, up to 61%, up to 62%, up to 63%, up to 64%, up to 65%, up to 66%, up to 67%, up to 68%, up to 69%, or up to 70% % of the total CBD dose. In some embodiments, the third pulse-release component (C₃) may comprise up to 30%, up to 31%, up to 32%, up to 33%, up to 34%, up to 35%, up to 36%, up to 37%, up to 38%, up to 39%, or up to 40% of the total CBD dose.

In some embodiments, the immediate release (IR) formulation comprises a mixture of ingredients that allow the IR formulation to dissolve quickly and allow rapid release of the one or more cannabinoid API after oral administration to a subject or after placing in a simulated gastrointestinal environment. In some embodiments, the delayed release (DR) formulation comprises a mixture of ingredients that delay dissolution of the DR formulation and thereby delay release of the one or more cannabinoid API after oral administration to a subject or after placing in a simulated gastrointestinal environment.

In some embodiments, the immediate release (IR) formulation can comprise one or more pharmaceutically-acceptable excipients. In some embodiments, the one or more pharmaceutically-acceptable excipient can include, without limitation, microcrystalline cellulose, corn starch, pregelatinized starch, potato starch, rice starch, sodium carboxymethyl starch, hydroxypropylcellulose, hydroxypropylmethylcellulose, hydroxyethylcellulose, ethylcellulose, chitosan, hydroxychitosan, hydroxymethylatedchitosan, cross-linked chitosan, cross-linked hydroxymethyl chitosan, maltodextrin, mannitol, sorbitol, dextrose, maltose, fructose, glucose, levulose, sucrose, polyvinylpyrrolidone (PVP), an acrylic acid derivative (e.g., Carbopol, Eudragit, etc.), a polyethylene glycol (PEG) such a low molecular weight PEGs (PEG2000-10000) and high molecular weight PEGs (e.g., Polyox) with molecular weights above 20,000 daltons, or any combination thereof.

In some embodiments, the pharmaceutically-acceptable excipients may comprise isomalt.

In some embodiment, the pharmaceutically-acceptable excipients may comprise from 1% to 60% by weight of the immediate release (IR) formulation.

In some embodiments, the immediate release (IR) formulation may include, without limitation, one or more binders, one or more disintegrants, one or more lubricants, one or more flow aids, glidants, or any combination thereof.

In some embodiments, the immediate release (IR) formulation may include one or more pharmaceutically-acceptable surfactants. In some embodiments, the one or more pharmaceutically-acceptable surfactant may include, without limitation, sodium lauryl sulfate, sodium monoglycerate, sorbitan monooleate, polyoxyethylene sorbitan monooleate, glyceryl monostearate, glyceryl monooleate, glyceryl monobutyrate, a non-ionic surfactant such as the Pluronic line of surfactants, or any other material with surface active properties, or any combination thereof.

In some embodiments, the pharmaceutically-acceptable surfactants may comprise from 0.05% to 15% by weight of the immediate release (IR) formulation.

In some embodiments, the delayed-release (DR) formulation may include a core comprising the one or more cannabinoid API and one or more pharmaceutically-acceptable excipients and optionally one or more pharmaceutically-acceptable surfactants, and may further include delayed-release (DR) components comprising one or more polymers in an amount and configuration to delay release of the one or more cannabinoid API from the core.

In some embodiments, the one or more polymers of the delayed-release (DR) components are comprised in a layer coating the core, or are integrated within the core, or both

In some embodiments, the delayed release (DR) formulation may include, without limitation, one or more binders, one or more lubricants, one or more flow aids, or any combinations thereof. In some embodiments, the delayed release (DR) formulation may include a coating layer of one or more pH-dependent or non-pH-dependent polymers, one or more plasticizers, or both. Accordingly, in some embodiments, the delayed-release (DR) formulation may include a non-pH sensitive delayed-release formulation, a pH sensitive delayed-release formulation, or a combination thereof.

In some embodiments, the one or more polymers of the delayed-release (DR) components of the non-pH sensitive delayed-release formulation may include, without limitation, polyethylene glycol (PEG) with molecular weight above 4,000 daltons (e.g., Carbowax, Polyox), a wax such as white wax or bees wax, paraffin, an acrylic acid derivative (e.g., a Eudragit), propylene glycol, ethylcellulose, or any combination thereof. In some embodiments, the one or more polymers may comprise from 0.05% to 25% by weight of the delayed-release (DR) formulation.

In some embodiments, the one or more polymers of the delayed-release (DR) components of the pH sensitive delayed-release formulation may include, without limitation, cellulose acetate pthalate, Eudragit L, a pthalate salt of a cellulose derivative, or any combination thereof. In some embodiments, the one or more polymers may comprise from 4% to 25% by weight of the delayed-release (DR) formulation.

In some embodiments, the binder may include, but is not limited to, microcrystalline cellulose, corn starch, pregelatinized starch, potato starch, rice starch, sodium carboxymethyl starch, hydroxypropylcellulose, hydroxypropylmethylcellulose, hydroxyethylcellulose, ethylcellulose, chitosan, hydroxychitosan, hydroxymethylatedchitosan, cross-linked chitosan, cross-linked hydroxymethyl chitosan, maltodextrin, mannitol, sorbitol, dextrose, maltose, fructose, glucose, levulose, sucrose, polyvinylpyrrolidone (PVP), acrylic acid derivatives (Carbopol, Eudragit, etc.), polyethylene glycols, such a low molecular weight PEGs (PEG2000-10000) and high molecular weight PEGs (Polyox) with molecular weights above 20,000 daltons.

In some embodiments, binders may be present in a pulse-release component of the pulse-release dosage form in the range of 1.0 to 60% (W/W).

Various well-known ingredients that may be included in a pulse-release component of the pulse-release dosage form to aid in the dissolution of the API, or the breakdown of the pulse-release component after ingestion or administration may include, without limitation, one or more surfactants, such as sodium lauryl sulfate, sodium monoglycerate, sorbitan monooleate, sorbitan monooleate, polyoxyethylene sorbitan monooleate, glyceryl monostearate, glyceryl monooleate, glyceryl monobutyrate, a non-ionic surfactant such as the Pluronic line of surfactants, or any other material with surface active properties.

In some embodiments, the disintegrant may include, but is not limited to, sodium starch glycolate, corn starch, rice starch, gar gum, polyvinylpolypyrrolidone, croscarmellose, croscarmellose sodium, and hydroxypropylmethylcellulose, among others.

In some embodiments, disintegrants may be present in a pulse-release component of the pulse-release dosage form in the range of 0.05-15% (W/W).

In some embodiments, the lubricant may include, but is not limited to, magnesium stearate, calcium stearate, and stearic acid, among others.

In some embodiments, the lubricants may be present in a pulse-release component of the pulse-release dosage form in the range of 0.05-5% (W/W).

In some embodiments, the flow aid may include, but is not limited to, colloidal silicone dioxide, silicon dioxide, and magnesium stearate, among others.

In some embodiments, the flow aids may be present in a pulse-release component of the pulse-release dosage form in the range of 0.05-0.5% (W/W).

In some embodiments, the polymer coating may include, but is not limited to, one or more pH-dependent or non-pH-dependent excipients. Examples of non-pH dependent polymers include ethyl cellulose, hydroxypropylmethyl cellulose, hydroxyethyl cellulose, hydroxypropyl cellulose, carboxymethyl cellulose, copolymer of ethyl acrylate, methyl methacrylate (e.g., Eudragit RS), among others. Examples of pH-dependent excipients include methacrylic acid copolymers, hydroxypropylmethyl cellulose acetate succinate, hydroxypropylmethyl cellulose phthalate, and cellulose acetate phthalate, among others. A pH-dependent polymer coating may also include a pore former, such as povidone, polyethylene glycol, hydroxypropyl cellulose, hydroxypropylmethyl cellulose, among others, sugars such as sucrose, mannitol, lactose, and salts, such as sodium chloride, sodium citrate, among others. In some embodiments, the polymer coating may include a plasticizer, such as acetylated citrated esters, acetylated glycerides, castor oil, citrate esters, dibutylsebacate, glyceryl monostearate, diethyl phthalate, glycerol, medium chain triglycerides, propylene glycol, and polyethylene glycol. The coating may also include one or more additional excipients, such as lubricants (e.g., magnesium stearate, talc among others). The coating can be applied using conventional coating techniques such as fluidized bed coating, pan coating, among others.

In certain embodiments, the pH-dependent polymer can be a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly (methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers. In certain other embodiments, the acrylic polymer is comprised of one or more ammonia methacrylate copolymers. Ammonio methacrylate copolymers are well known in the art, and are described as fully polymerized copolymers of acrylic and methacrylic acid esters with a low content of quaternary ammonium groups. In still other embodiments, the acrylic polymer may be an acrylic resin lacquer such as a Eudragit®. In further embodiments, the acrylic polymer comprises a mixture of two acrylic resin lacquers commercially available from Evonik/Rohm Pharma under the trade names Eudragit® RL30D and Eudragit® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. Eudragit® S-100 and Eudragit® L-100 are also suitable for use herein. The code designations RL (high permeability) and RS (low permeability) refer to the permeability properties of these agents. Eudragit® mixtures are insoluble in water and in digestive fluids. However, multi-particulate systems formed to include the same are swellable and permeable in aqueous solutions and digestive fluids. The polymers described above such as Eudragit® RL/RS may be mixed together in any desired ratio in order to ultimately obtain an extended release formulation having a desirable dissolution profile. One skilled in the art will recognize that other acrylic polymers may also be used, such as, for example, Eudragit® L.

In some embodiments, a delayed release component may comprise an enteric coating, for example, without limitation, methacrylic acid-ethyl acrylate copolymer (1:1), e.g. Vivacoat® (JRS Pharma).

In some embodiments, the polymer coatings may be present in a pulse-release component of the pulse-release dosage form in the range of 0.5-35% (W/W).

In some embodiments, the pulse-release dosage form may include API coated cores or substrates. In some embodiments, the cores or substrates may comprise non-pareils or sugar spheres.

In some embodiments, the pulse-release dosage form may optionally comprise a seal coat. In some embodiments, a DR pulse-release component of the pulse-release dosage form may be coated with a polymer coating by fluidized bed coating.

In some embodiments, a pulse-release component of the present disclosure can be prepared by any suitable method known in the art, such as mixing the ingredients in a suitable pharmaceutical mixer or granulator such as a planetary mixer, high-shear granulator, fluid bed granulator, or extruder, in the presence of water or other solvent, or in a hot melt process. If water or other solvent is used, the blend may be dried in a suitable pharmaceutical drier, such as a vacuum oven or forced-air oven. After allowing the product to cool, the product may be sieved or granulated, and compressed using a suitable tablet press, such as a rotary tablet press.

A pharmaceutical formulation of the present disclosure may be further processed into a solid dosage form suitable for oral administration, such as a pill, tablet or capsule. In some embodiments, the pharmaceutical formulation of the present disclosure may be provided in oral dosage forms such as a syrup, film, orally-disintegrating tablet, a liquid solution or suspension (e.g., drink or syrup), a powder, or liquid or solid crystals, or a paste.

In some embodiments, each of the pulse-release components is formulated as a plurality of particles, or pellets. In some embodiments, the particles or pellets may be contained in a capsule, formed as a tablet, or suspended in a liquid.

Example methods of formulating example pulse-release oral dosage form of the present disclosure is described in Examples 1-65.

### C. Method of Treating an Inflammatory Gastrointestinal Disorder

In some embodiments, the present disclosure provides a method of treating an inflammatory gastrointestinal disorder in a subject in need thereof, the method comprising administering to the subject a therapeutically effective amount of an oral pulse-release dosage form of the present disclosure.

In some embodiments, the inflammatory gastrointestinal disorder comprises inflammation of the upper gastrointestinal tract, the lower gastrointestinal tract, or both.

The pharmaceutical compositions of the disclosure are designed to provide novel release profiles of the one or more cannabinoid API that provide a timed, targeted, and/or prolonged therapeutic effect when taken e.g. once a day.

In some embodiments described herein, the present disclosure provides an oral pulse-release dosage form configured for timed release of the one or more cannabinoid API in the subject's gastrointestinal tract. In some embodiments, the timing of release from a pulse-release component can be such that a portion of the total dose of the one or more cannabinoid API begins to be released or has a peak release rate at a particular region of the gastrointestinal tract, such as, for example, in the subject's esophagus, stomach, duodenum, jejunum, ileum, colon, rectum or anal canal.

Typical gastrointestinal transit times are, for example, gastric emptying 90 minutes, small intestine transit time 4-6 hours, colon arrival times approximately 6-8 hours.

The human gastrointestinal tract includes several distinct parts, including upper (more proximal to the oral opening) and lower (more distal from the oral opening) portions of the gastrointestinal tract, including the esophagus, stomach, duodenum, jejunum, ileum, colon, rectum and anal canal. The human gastrointestinal tract may be understood as a long tube passing from the oral opening to the anal opening. The main function is to supply the body with water, electrolytes and nutrients from ingested food. The main sources of nutrients are carbohydrates, proteins and fats, which in general cannot be absorbed in the form they are ingested. First, nutrients have to be broken down into small enough compounds. The process of digestion and absorption is carried out in a stepwise fashion as the food passes down the different parts of the gastrointestinal tract. Likewise, orally administered drugs typically also pass through the human gastrointestinal tract in a stepwise fashion.

The most proximal (closest to the oral opening) and widest part of the small intestine is the duodenum. Starting at the pylorus of the stomach, the duodenum ends at the duodenojejunal junction and measures about 25 cm long. It receives partly digested food (chyme) from the stomach and bile and pancreatic fluids from the pancreaticobiliary duct. After entering the duodenum, the acidic contents from the stomach is neutralized by secretion from the intestine and pancreas. Enzymes secreted from the pancreas starts the digestion of lipids, carbohydrates and proteins to enable absorption. The small intestine includes the jejunum and ileum and measures about 6 meters and absorbs nutrition, water and electrolytes.

The large intestine, also known as the large bowel, is the last part of the gastrointestinal tract and of the digestive system in vertebrates. Water is absorbed here and the remaining waste material is stored as feces before being removed by defecation, and is the site in which flora-aided (largely bacterial) fermentation of some of the indigestible food matter occurs. The large intestine includes, from proximal to distal end, the cecum, colon, rectum, and anal canal. The colon is the largest portion of the large intestine. The colon is divided into four parts, from proximal end to distal end, including the ascending colon, transverse colon, descending colon and sigmoid colon. In humans, the most proximal portion of the large intestine begins in the right iliac region of the pelvis, just at or below the waist, where it is joined to the end of the small intestine at the cecum, via the ileocecal valve. It then continues as the colon ascending the abdomen, across the width of the abdominal cavity as the transverse colon, and then descending to the rectum and its endpoint at the anal canal. Overall, in humans, the large intestine is about 1.5 m long.

The oral pulse-release dosage forms described herein may include a therapeutically effective amount of the one or more cannabinoid API, which amount will vary with the cannabinoid API used in the oral pulse-release dosage form, the inflammatory gastrointestinal disorder to be treated, and the number of times that the oral pulse-release dosage form is to be administered to a subject in a day. These may also vary in respect to the location of the inflammation in the gastrointestinal tract.

If the inflammation is predominately in the upper portion of the gastrointestinal tract, for example, an oral pulse-release dosage form comprising an immediate release (IR) formulation and a delayed release (DR) targeting the upper gastrointestinal tract may be sufficient to effectively treat the inflammation. If the inflammation is predominately in the lower gastrointestinal tract, two, three or four delayed release (DR) formulations targeting the lower GI tract may be sufficient to effectively treat the inflammation. If the inflammation is spread throughout the GI tract, as many as four delayed release (DR) formulations in addition to an immediate release (IR) formulation may be needed to effectively treat the inflammation. In some instances, one or two delayed and/or sustained release formulations may be included in the oral pulse-release dosage form to provide adequate coverage of the gastrointestinal tract. Examples of delayed release and sustained release formulations are provided in the Examples herein.

For example, in some embodiments, for an oral pulse-release dosage form comprising a first pulse-release component (C₁) comprising an immediate release (IR) formulation, and a second pulse-release component (C₂), and a third pulse-release component (C₃) each comprising a delayed-release (DR) formulation, the oral pulse-release dosage form may be configured such that the first pulse-release component (C₁) may have a PRR₁ when the first pulse-release component (C₁) arrives in the subject's stomach, the second pulse-release component (C₂) may have a PRR₂ when the second pulse-release component (C₂) arrives in the subject's jejunum, and third pulse-release component (C₃) may have a PRR₃ when the third pulse-release component (C₃) arrives in the subject's colon. Other configurations of the various release components (e.g., first, second, third, fourth, or fifth release components described herein) of the oral pulse-release dosage forms described herein for targeting release of one or more cannabinoid API for release into various regions of the gastrointestinal tract are identifiable by persons of ordinary skill in the art upon reading the present disclosure.

In some embodiments, the inflammatory gastrointestinal disorder comprises irritable bowel syndrome, inflammatory bowel disease, Crohn's disease, ulcerative colitis, perianal fistulizing disease (e.g., ulcerative colitis-related or Crohn's disease-related), left sided colitis, ulcerative proctitis, Gastro-Esophogeal Reflux Disease (GERD), an inflammatory gastrointestinal disorder following Gall bladder removal, an undiagnosed inflammatory gastrointestinal disorder, or any combination thereof. Identification of a site, or a primary site, of inflammation within one or more regions of the gastrointestinal tract of a subject can be performed using various medical or veterinary diagnostic methods known in the art.

In some embodiments, administration of an oral pulse-release dosage form described herein may result in treatment of the inflammatory gastrointestinal disorder within 6 hours post-dose, within 12 hours post-dose, within 24 hours post-dose, or greater than 24 hours post-dose.

In some embodiments, administration of an oral pulse-release dosage form described herein may result in treating one or more symptoms of the inflammatory gastrointestinal disorder for up to 1 hour per day, up to 2 hours per day, up to 3 hours per day, up to 4 hours per day, up to 5 hours per day, up to 6 hours per day, up to 7 hours per day, up to 8 hours per day, up to 9 hours per day, up to 10 hours per day, up to 11 hours per day, up to 12 hours per day, up to 13 hours per day, up to 14 hours per day, up to 15 hours per day, up to 16 hours per day, up to 17 hours per day, up to 18 hours per day, up to 19 hours per day, up to 20 hours per day, up to 21 hours per day, up to 22 hours per day, up to 23 hours per day, up to 24 hours per day, or for longer than 24 hours. The one or more symptoms may include, without limitation, pain, bloating, diarrhea, loss of appetite, or any combination thereof.

The term “subject” as used herein refers to a warm blooded animal such as a mammal which is treated for an inflammatory gastrointestinal disorder that causes at least one symptom. It is understood that at least humans, dogs, cats, and horses are within the scope of the meaning of the term. In some embodiments, the subject is a human. In particular, the subject may be in need of treatment for an inflammatory gastrointestinal disorder.

As used herein, the term “treat” or “treatment”, or a derivative thereof, contemplates partial or complete amelioration of at least one symptom associated with the inflammatory gastrointestinal disorder in the subject.

### EXAMPLES

The present examples are provided for illustrative purposes only. They are not intended to and should not be interpreted to encompass the full breadth of the disclosure.

### Examples 1-15. Example Formulations of an Immediate Release Component

An example immediate release component may be formulated as follows:

Formulate the composition by mixing the ingredients in a suitable pharmaceutical mixer or granulator such as a planetary mixer, high-shear granulator, fluid bed granulator, or extruder, in the presence of water or other solvent, or in a dry blend. If water or other solvent was used, dry the blend in a suitable pharmaceutical drier, such as a vacuum over or forced-air oven. The product may be sieved or granulated, and compressed using a suitable tablet press, such as a rotary tablet press. In each of the following examples, the term “Cannabinoid” refers to the total cannabinoid(s) to be used in the immediate release formulations. These examples may have CBG, CBD and THC individually, or in any combination thereof. The percentages referred to in the following examples refers to Ingredient Conc. (% W/W).

### Example 1

Cannabinoid 65% (W/W)
Microcrystalline cellulose 20%
Povidone 10%
Croscarmellose sodium 5%

### Example 2

Cannabinoid 55% (W/W)
Microcrystalline cellulose 25%
Povidone 10%
Croscarmellose sodium 10%

### Example 3

Cannabinoid 65% (W/W)
Microcrystalline cellulose 20%
Hydroxypropylcellulose 10%
Croscarmellose sodium 5%

### Example 4

Cannabinoid 75% (W/W)
Polyethylene glycol 4000 10%
Polyethylene glycol 2000 10%
Hydroxypropylcellulose 5%

### Example 5

Cannabinoid 75% (W/W)
Polyethylene glycol 8000 20%
Polyvinylpyrrolidone 5%

### Example 6

Cannabinoid 65% (W/W)
Microcrystalline cellulose 20%
Hydroxypropylcellulose 10%
Croscarmellose sodium 5%

### Example 7

Cannabinoid 75% (W/W)
Micro crystalline cellulose 15%
Hydroxypropylcellulose 5%
Cannabinoid 5%

### Example 8

Cannabinoid 75% (W/W)
Polyethylene glycol 4000 10%
Polyethylene glycol 2000 10%
Hydroxypropylcellulose 5%

### Example 9

Cannabinoid 75% (W/W)
Polyethylene glycol 8000 20%
Polyvinylpyrrolidone 5%

### Example 10

Cannabinoid 65% (W/W)
Microcrystalline cellulose 20%
Hydroxypropylcellulose 10%
Croscarmellose sodium 5%

### Example 11

Cannabinoid 75% (W/W)
Microcrystalline cellulose 15%
Hydroxypropylcellulose 5%
Croscarmellose sodium 5%

### Example 12

Cannabinoid 75% (W/W)
Polyethylene glycol 4000 10%
Polyetheylene glycol 2000 10%
Hydroxypropylcellulose 5%

### Example 13

Cannabinoid 75% (W/W)
Polyethylene glycol 8000 20%
Polyvinylpyrrolidone 5%

### Example 14

Cannabinoid 75% (W/W)
Polyethylene glycol 4000 10%
Polyethylene glycol 2000 10%
Hydroxypropylcellulose 5%

### Example 15

Cannabinoid 75% (W/W)
Polyethylene Glycol 4000 20%
Polyvinylpyrrolidone 5%

### Examples 16-30. Example Formulations of a Non-pH Sensitive Delayed Release Component

An example non-pH Sensitive Delayed Release Component may be formulated as follows:

Formulate the composition by mixing the ingredients in a suitable pharmaceutical mixer or granulator such as a planetary mixer, high-shear granulator, fluid bed granulator, or extruder, in the presence of water or other solvent, or in a hot melt process. If water or other solvent was used, dry the blend in a suitable pharmaceutical drier, such as a vacuum over or forced-air oven. Allow the product to cool, the product may be sieved or granulated, and compressed using a suitable tablet press, such as a rotary tablet press. In each of the following examples, the term “Cannabinoid” refers to the total cannabinoid(s) to be used in the non-pH sensitive delayed release formulations. These examples may have CBG, CBD and THC individually, or in any combination thereof. The percentages referred to in the following examples refers to Ingredient Conc. (% W/W).

### Example 16

Cannabinoid 65% (W/W)
Microcrystalline cellulose 20%
Polyox 10 Croscarmellose sodium 5%

### Example 17

Cannabinoid 55% (W/W)
Microcrystalline cellulose 25%
Polyox 10%
Glyceryl monooleate 10%

### Example 18

Cannabinoid 65% (W/W)
Polyox 20%
Hydroxypropylcellulose 10%
Croscarmellose sodium 5%

### Example 19

Cannabinoid 75% (W/W)
Polyethylene glycol 4000 10%
Polyethylene glycol 2000 10%
Eudragit RL 30D 5%

### Example 20

Cannabinoid 75% (W/W)
Polyethylene glycol 8000 20%
Ethylcellulose 5%

### Example 21

Cannabinoid 70% (W/W)
Polyox 20%
Hydroxypropylcellulose 5%
Croscarmellose sodium 5%

### Example 22

Cannabinoid 75% (W/W)
Polyox 15%
Hydroxypropylcellulose 5%
Ethylcellulose 5%

### Example 23

Cannabinoid 75% (W/W)
Polyethylene glycol 4000 10%
Polyethylene glycol 2000 10%
Eudragit RL 30D 5%

### Example 24

Cannabinoid 80% (W/W)
Polyethylene glycol 8000 10%
Polyvinylpyrrolidone 5%
Eudragit R 30D 5%

### Example 25

Cannabinoid 65% (WW)
Polyethylene glycol 4000 20%
Hydroxypropylcellulose 10%
Eudragit RL 30D 5%

### Example 26

Cannabinoid 75% (W/W)
Microcrystalline cellulose 15%
Hydroxypropylcellulose 5%
Ethylcellulose 5%

### Example 27

Cannabinoid 80% (W/W)
Polyethylene glycol 4000 10%
Polyethylene glycol 2000 5%
Eudragit RL 30D 5%

### Example 28

Cannabinoid 75% (W/W)
Polyethylene glycol 8000 20%
Ethylcellulose 5%

### Example 29

Cannabinoid 75% (W/W)
Polyethylene glycol 4000 10%
Polyethylene glycol 2000 10%
Eudragit RL 30D 5%

### Example 30

Cannabinoid 75% (W/W)
Polyethylene glycol 8000 20%
Ethylcellulose 5%

### Examples 31-45. Example Formulations of an Enteric Release Component

An example Enteric Release Component may be formulated as follows:

Formulate the ingredients by mixing the ingredients in a suitable pharmaceutical mixer or granulator such as a planetary mixer, high-shear granulator, fluid bed granulator, or extruder, in the presence of water or other solvent, or in a hot melt process. If water or other solvent was used, dry the blend in a suitable pharmaceutical drier, such as a vacuum over or forced-air oven. Allow the product to cool, the product may be sieved or granulated, and compressed using a suitable tablet press, such as a rotary tablet press. In each of the following examples, the term “Cannabinoid” refers to the total cannabinoid(s) to be used in the enteric release formulations. These examples may have CBG, CBD and THC individually, or in any combination thereof. The percentages referred to in the following examples refers to Ingredient Conc. (% W/W).

### Example 31

Cannabinoid 65% (W/W)
Microcrystalline cellulose 20%
Cellulose Acetate Pthalate 15%

### Example 32

Cannabinoid 55% (W/W)
Microcrystalline cellulose 25%
Cellulose Acetate Pthalate 10%
Hydroxypropylmethylcellulose 10%

### Example 33

Cannabinoid 65% (W/W)
Polyox 20%
Hydroxypropylcellulose pthalate 10%
Eudragit L30D 5%

### Example 34

Cannabinoid 75% (W/W)
Polyethylene glycol 2000 10%
Eudragit L30D 10%
Eudragit RL 30D 5%

### Example 35

Cannabinoid 40% (W/W)
Microcrystalline Cellulose 40%
Cellulose Acetate Pthalate 10%

### Example 36

Cannabinoid 70% (W/W)
Hydroxypropylcellulose pthalate 15%
Croscarmellose sodium 10%

### Example 37

Cannabinoid 70% (W/W)
Eudragit E30D 15%
Hydroxypropylcellulose 10%
Ethylcellulose 5%

### Example 38

Cannabinoid 75% (W/W)
Polyethylene glycol 2000 10%
Eudragit E 30D 15%

### Example 39

Cannabinoid 40% (W/W)
Lactose 50%
Eudragit L 30D 10%

### Example 40

Cannabinoid 65% (W/W)
Microcrystalline Cellulose 20%
Eudragit L 30D 10%

### Example 41

Cannabinoid 75% (W/W)
Microcrystalline Cellulose 15%
Hydroxypropylcellulose pthalate 10%

### Example 42

Cannabinoid 80% (W/W)
Lactose 10%
Eudragit L 30D 10%

### Example 43

Cannabinoid 70% (W/W)
Polyethylene glycol 4000 20%
Cellulose acetate pthalate 10%

### Example 44

Cannabinoid 60% (W/W)
Polyethylene glycol 2000 10%
Lactose 20%
Eudragit L 30D 10%

### Example 45

Cannabinoid 70% (W/W)
Microcrystalline cellulose 20%
Cellulose acetate pthalate 10%

### Examples 46-55. Example Formulations of an Sustained Release Component

An example Sustained Release Component may be formulated as follows:

Formulate the composition by mixing the ingredients in a suitable pharmaceutical mixer or granulator such as a planetary mixer, high-shear granulator, fluid bed granulator, or extruder, in the presence of water or other solvent, or in a hot melt process. If water or other solvent was used, dry the blend in a suitable pharmaceutical drier, such as a vacuum over or forced-air oven. Allow the product to cool, the product may be sieved or granulated, and compressed using a suitable tablet press, such as a rotary tablet press. In each of the following examples, the term “Cannabinoid” refers to the total cannabinoid(s) to be used in the sustained release formulations. These examples may have CBG, CBD and THC individually, or in any combination thereof. The percentages referred to in the following examples refers to Ingredient Conc. (% W/W).

### Example 46

Cannabinoid 65% (W/W)
Ethylcellulose 20%
Polyox 10%
Hydroxypropylmethylcellulose 5%

### Example 47

Cannabinoid 55% (W/W)
Lactose 25 Polyox 10%
Glyceryl monooleate 10%

### Example 48

Cannabinoid 70% (W/W)
Polyox 20%
Hydroxypropylcellulose 10%

### Example 49

Cannabinoid 75% (W/W)
Lactose 15%
Hydroxypropylcellulose 5%
Ethylcellulose 5%

### Example 50

Cannabinoid 75% (W/W)
Polyethylene glycol 4000 10%
Lactose 10%
Eudragit RL 30D 5%

### Example 51

Cannabinoid 80% (W/W)
Polyethylene glycol 8000 10%
Hydroxypropylmethylcellulose 5%
Eudragit RS 30D 5%

### Example 52

Cannabinoid 75% (W/W)
Hydroxyethylcellulose 10%
Polyethylene glycol 4000 10%
Hydroxypropylcellulose 5%

### Example 53

Cannabinoid 75% (W/W)
Lactose 10%
Povidone (PVP) 10%
Polyethylene glycol 2000 5%

### Example 54

Cannabinoid 75% (W/W)
Polyethylene glycol 4000 10%
Povidone (PVP) 10%
Hydroxypropylcellulose 5%

### Example 55

Cannabinoid 75% (W/W)
Lactose 15%
Polyethylene glycol 4000 5%
Polyvinylpyrrolidone 5%

Examples 56-65 provide example formulations of pulse-release cannabinoid oral dosage forms of the present disclosure.

### Example 56. Example Three-Pulse Formulation

An example three-pulse formulation may be prepared as follows:

### Cannabinoid Matrix Pellet Formulation and Preparation Procedure (Immediate Release)

### A. Pellet Formulation

The composition of the cannabinoid matrix pellets provided in Table 1.

TABLE 1
Composition of Cannabinoid Pellets

| Component | Percentage (%) |
|---|---|
| Cannabinoid | 50 |
| Avicel PH 101 | 20 |
| Lactose | 20 |
| PVP K29/32* | 10 |
| Purified Water | |
| Total | 100 |

*PVP K29/32 was added as a 20% w/w aqueous solution during wet massing.

### B. Preparation Procedure for Cannabinoid Matrix Pellets

Blend cannabinoid and Avicel® PH 101 using a Robot Coupe high shear granulator.
Add 20% Povidone K29/32 binder solution slowly into the powder blend under continuous mixing.
Extrude the wet mass using an LCI Bench Top Granulator. The diameter of the screen of the Bench Top Granulator was 1.0 mm.
Spheronize the extrudate using a Model SPH20 Caleva Spheronizer.
Dry the spheronized pellets at 50.degree. C. overnight.
Pellets between 16 and 30 Mesh were collected for further processing.

### Preparation of an Eudragit® L30D-55 Aqueous Coating Dispersion

### A. Dispersion Formulation

The composition of the aqueous Eudragit L30D-55 dispersion applied to the cannabinoid matrix pellets is provided below in Table 2.

TABLE 2
Eudragit. RTM. L 30 D-55 Aqueous Dispersion

| Component | Percentage (%) |
|---|---|
| Eudragit. RTM. L 30 D-55 | 55.0 |
| Triethyl Citrate | 1.6 |
| Talc | 8.0 |
| Purified Water | 35.4 |
| Solids Content | 25.5 |
| Polymer Content | 15.9 |

### B. Preparation Procedure for an Eudragit® L30D-55 Aqueous Dispersion

Suspend triethyl citrate and talc in deionized water.
The TEC/talc suspension is then homogenized using a PowerGen 700 high shear mixer.
Add the TEC/talc suspension slowly to the Eudragit® L30D-55 latex dispersion while stirring.
Allow the coating dispersion to stir for one hour prior to application onto the cannabinoid matrix pellets.

### Preparation of an Eudragit® S 100 Aqueous Coating Dispersion

### A. Dispersion Formulation

The composition of the aqueous Eudragit® S 100 dispersion applied to the cannabinoid matrix pellets is provided below in Table 3.

TABLE 3
Eudragit. RTM. S 100 Aqueous Coating Dispersion

| | |
|---|---|
| Component | Percentage (%) |

Part A

| | |
|---|---|
| Eudragit. RTM. S 100 | 12.0 |
| 1N Ammonium Hydroxide | 6.1 |
| Triethyl Citrate | 6.0 |
| Purified Water | 65.9 |

Part B

| | |
|---|---|
| Talc | 2.0 |
| Purified Water | 8.0 |
| Solid Content | 20.0 |
| Polymer Content | 12.0 |

### B. Preparation Procedure for an Eudragit® S 100 Aqueous Dispersion

### Part I:

(i) Dispense Eudragit® S 100 powder in deionized water with stirring.
(ii) Add ammonium hydroxide solution drop-wise into the dispersion with stirring.
(iii) Allow the partially neutralized dispersion to stir for 60 minutes.
(iv) Add triethyl citrate drop-wise into the dispersion with stirring. Stir for about 2 hours prior to the addition of Part B.

### Part II:

(i) Disperse talc in the required amount of water
(ii) Homogenize the dispersion using a PowerGen 700D high shear mixer.
(iii) Part B is then added slowly to the polymer dispersion in Part A with a mild stirring.

### Coating Conditions for the Application of Aqueous Coating Dispersions

The following coating parameters were used to coat matrix pellets with each of the Eudragit® L30D-55 and Eudragit® S 100 aqueous film coating.

The following coating conditions were used.

### Coating Equipment

STREA 1™ Table Top Laboratory Fluid Coating Equipment Bed Coater
Spray nozzle diameter 1.0 mm
Material Charge 300 gram
Inlet Air Temperature 40 to 45.degree. C.
Outlet Air Temperature 30 to 33.degree. C.
Atomization Air Pressure 1.8 Bar
Pump Rate 2 gram per minute
(i) Coat matrix pellets with L30 D-55 dispersion such that you apply 12% coat weight gain to the pellets.
(ii) Coat matrix pellets with S 100 dispersion such that you apply 20% coat weight gain to the pellets.
   Encapsulation of the Cannabinoid Pellets: Pellets are Filled into Size 00 Hard Gelatin Capsules at a Ratio of 30%:30%:40%:
Immediate-release matrix pellets uncoated, L30 D-55 coated pellets and S100 coated pellets respectively.
The capsule is filled with the three different pellets to achieve a total dose of 375 mg/capsule.

### Example 57. Example Three-Pulse Formulation

An example three-pulse formulation may be prepared as follows:

### Cannabinoid Pellet Formulation and Preparation Procedure

### Pellet Formulations for Subsequent Coating

The composition of the Cannabinoid matrix pellets provided in Table 4.

TABLE 4
Composition of Cannabinoid Matrix Pellets

| Component | Percentage (%) |
|---|---|
| Cannabinoid | 92 |
| Avicel PH 101 | 7.0 |
| Hydroxypropyl methylcellulose, NF* | 1.0 |
| Total | 100 |

*Hydroxypropyl methylcellulose was added as a 2.9% w/w aqueous solution during wet massing.

### Preparation Procedure for Cannabinoid Matrix Pellets

Blend Cannabinoid and Avicel® PH 101 using a low shear blender.
Add the hydroxypropyl methylcellulose binder solution slowly into the powder blend under continuous mixing.
Extrude the wet mass using an LCI Bench Top Granulator. The diameter of the screen of the Bench Top Granulator is 0.8 mm.
Spheronize the extrudate using a QJ-230 Spheronizer using a small cross section plate.
Dry the spheronized pellets at 60.degree. C. using a fluid bed dryer until the exhaust temperature reaches 40.degree. C.
Pellets between 20 and 40 Mesh were collected for further processing.

### Preparation of an Eudragit® L30D-55 Aqueous Coating Dispersion

### Dispersion Formulation

The composition of the aqueous Eudragit L30D-55 dispersion applied to the cannabinoid matrix pellets is provided below in Table 5.

TABLE 5
Eudragit. RTM. L 30 D-55 Aqueous Coating Dispersion

| Component | Percentage (%) |
|---|---|
| Eudragit. RTM. L 30 D-55 | 41.6 |
| Triethyl Citrate | 2.5 |
| Talc | 5.0 |
| Purified Water | 50.9 |
| Solids Content | 20.0 |
| Polymer Content | 12.5 |

### Preparation Procedure for an Eudragit® L30D-55 Aqueous Dispersion

Suspend triethyl citrate and talc in deionized water.
The TEC/talc suspension is mixed using laboratory mixer.
Add the TEC/talc suspension from slowly to the Eudragit® L30D-55 latex dispersion while stirring.
Allow the coating dispersion to stir for one hour prior to application onto the cannabinoid matrix pellets.

### Preparation of an Eudragit® S 100 Aqueous Coating Dispersion

### Dispersion Formulation

The composition of the aqueous Eudragit® S 100 dispersion applied to the Cannabinoid matrix pellets is provided below in Table 6.

TABLE 6
Eudragit. RTM. S 100 Aqueous Coating Dispersion

| | |
|---|---|
| Component | Percentage (%) |

Part A

| | |
|---|---|
| Eudragit. RTM. S 100 | 10.0 |
| 1N Ammonium Hydroxide | 5.1 |
| Triethyl Citrate | 5.0 |
| Water | 64.9 |

Part B

| | |
|---|---|
| Talc | 5.0 |
| Water | 10.0 |
| Solid Content | 25.0 |
| Polymer Content | 10.0 |

### Preparation Procedure for an Eudragit® S 100 Aqueous Dispersion

### Part A:

Dispense Eudragit® S 100 powder in deionized water with stirring.
Add ammonium hydroxide solution drop-wise into the dispersion with stirring.
Allow the partially neutralized dispersion to stir for 60 minutes.
Add triethyl citrate drop-wise into the dispersion with stirring and let stir overnight prior to the addition of Part B.

### Part B:

Disperse talc in the required amount of water
Stir the dispersion using an overhead laboratory mixer.
Part B is then added slowly to the polymer dispersion in Part A with a mild stirring.

### Coating Conditions for the Application of Aqueous Coating Dispersions

The following coating parameters were used for both the Eudragit® L30D-55 and Eudragit® S 100 aqueous film coating processes.

### Coating Equipment

STREA 1™ Table Top Laboratory Fluid Bed Coater
Spray nozzle diameter 1.0 mm
Material Charge 300 gram
Inlet Air Temperature 40 to 45.degree. C.
Outlet Air Temperature 30 to 33.degree. C.
Atomization Air Pressure 1.8
Bar Pump Rate 2-6 gram per minute
Coat matrix pellets with L30 D-55 dispersion such that you apply 20% coat weight gain to the pellets.
Coat matrix pellets with S100 dispersion such that you apply 37% coat weight gain to the pellets.

### Preparation of Cannabinoid Granulation (Immediate Release Component) for Tableting

TABLE 7
Composition of Cannabinoid Granulation

| Component | Percentage (%) |
|---|---|
| Cannabinoid powder | 92 |
| Avicel PH 101 | 7.0 |
| Hydroxypropyl methylcellulose, NF* | 1.0 |
| Total | 100 |

*Hydroxypropyl methylcellulose was added as a 2.9% w/w aqueous solution during wet massing.

Blend Cannabinoid and Avicel® PH 101 using a low shear blender. 57.8.2 Add the hydroxypropyl methylcellulose binder solution slowly into the powder blend under continuous mixing. Dry the granulation at 60.degree. C. using a fluid bed dryer until the exhaust temperature reaches 40.degree. C. Granules between 20 and 40 Mesh are collected for further processing.

### Tableting of the Cannabinoid Pellets

TABLE 8
Composition of Cannabinoid Tablets

| Component | Percentage (%) |
|---|---|
| Cannabinoid granules | 32.5 |
| Avicel PH 200 | 5.0 |
| Cannabinoid L30D-55 coated pellets | 30 |
| Cannabinoid S 100 coated pellets | 30 |
| Colloidal silicon dioxide | 1.5 |
| Magnesium stearate | 1.0 |
| Total | 100 |

Blend the Cannabinoid granules, Avicel PH-200, Cannabinoid pellets and colloidal silicon dioxide for 15 minutes in a tumble blender.
Add the magnesium stearate to the blender, and blend for 5 minutes.
Compress the blend on a rotary tablet press.
The fill weight should be adjusted to achieve a 500 mg dose tablet.

### Example 58. Example Three-Pulse Formulation

An example three-pulse formulation may be prepared as follows:

### Cannabinoid Pellet Formulation and Preparation Procedure

### Pellet Formulation

The composition of the cannabinoid matrix pellets provided in Table 1.

TABLE 9
Composition of Cannabinoid Pellets

| Component | Percentage (%) |
|---|---|
| Cannabinoid | 50.6 |
| Lactose monohydrate, spray dried | 32.1 |
| Silicified microcrystalline cellulose | 14.6 |
| Polyoxyl | 35 |
| Castor Oil* | 1.7 |
| Hydroxypropyl methylcellulose* | 1.0 |
| Total | 100 |

*Hydroxypropyl methylcellulose and Polyoxyl 35 were added as an 8.7% w/w aqueous solution during wet massing.

### Preparation Procedure for Cannabinoid Matrix Pellets

Blend cannabinoid, silicified microcrystalline cellulose and lactose monohydrate using a Robot Coupe high shear granulator.
Prepare the binder solution by adding the Polyoxyl to the purified water while stirring. After that is mixed, slowly add the hydroxypropyl methylcellulose and continue to stir until a solution is achieved.
Add binder solution slowly into the powder blend under continuous mixing.
Granulate the powders in the high shear granulator with the binder solution.
Extrude the wet mass using an LCI Bench Top Granulator. The diameter of the screen of the Bench Top Granulator was 1.2 mm.
Spheronize the extrudate using a Model SPH20 Caleva Spheronizer.
Dry the spheronized pellets at 50.degree. C. overnight.
Pellets between 18 and 30 Mesh were collected for further processing.

### Preparation of an Eudragit® L30 D-55 Aqueous Coating Dispersion

### Dispersion Formulation

The composition of the aqueous Eudragit L30D-55 dispersion applied to the cannabinoid matrix pellets is provided below in Table 10.

TABLE 10
Eudragit. RTM. L 30 D-55 Aqueous Coating Dispersion

| Component | Percentage (%) |
|---|---|
| Eudragit. RTM. L 30 D-55 | 40.4 |
| Triethyl Citrate | 1.8 |
| Talc | 6.1 |
| Water | 51.7 |
| Solids Content | 20.0 |
| Polymer Content | 12.1 |

### Preparation Procedure for an Eudragit® L30 D-55 Aqueous Dispersion

Suspend triethyl citrate and talc in deionized water.
The TEC/talc suspension is then homogenized using a PowerGen 700 high shear mixer.
Add the suspension from 4.2.2 slowly to the Eudragit® L30 D-55 latex dispersion while stirring.
Allow the coating dispersion to stir for one hour prior to application onto the cannabinoid matrix pellets.

### Preparation of an Eudragit® S 100 Aqueous Coating Dispersion

### Dispersion Formulation

The composition of the aqueous Eudragit® S 100 dispersion applied to the cannabinoid matrix pellets is provided below in Table 11.

TABLE 11
Eudragit. RTM. S 100 Aqueous Coating Dispersion

| | |
|---|---|
| Component | Percentage (%) |

Part A

| | |
|---|---|
| Eudragit. RTM. S 100 | 10.01 |
| N Ammonium Hydroxide | 5.1 |
| Triethyl Citrate | 5.0 |
| Water | 64.9 |

Part B

| | |
|---|---|
| Talc | 5.0 |
| Water | 10.0 |
| Solid Content | 25.0 |
| Polymer Content | 10.0 |

### Preparation Procedure for an Eudragit® S 100 Aqueous Dispersion

### Part A:

Dispense Eudragit® S 100 powder in deionized water with stirring.
Add ammonium hydroxide solution drop-wise into the dispersion with stirring.
Allow the partially neutralized dispersion to stir for 60 minutes
Add the triethyl citrate drop-wise to the dispersion and stir for 60 minutes prior to the addition of Part B.

### Part B:

Disperse talc in the required amount of water
Homogenize the dispersion using a PowerGen 700D high shear mixer.
Part B is then added slowly to the polymer dispersion in Part A with a mild stirring.

### Coating Conditions for the Application of Aqueous Coating Dispersions

The following coating parameters were used for coating the matrix pellets with each of the Eudragit® L30 D-55 and Eudragit® S 100 aqueous film coating.

### Coating Equipment

STREA 1™ Table Top Laboratory Fluid Bed Coater
Spray nozzle diameter 1.0 mm
Material Charge 300 gram
Inlet Air Temperature 40 to 45.degree. C.
Outlet Air Temperature 30 to 33.degree. C.
Atomization Air Pressure 1.6 Bar
Pump Rate 2 gram per minute

Coat matrix pellets with L30 D-55 dispersion such that you apply 20% coat weight gain to the pellets. Coat matrix pellets with S100 dispersion such that you apply 37% coat weight gain to the pellets.

Capsules were filled with the uncoated pellets, the L30D-55 coated pellets and S 100 coated pellets in weight percentages of 30%:30%:40%, respectively to provide 250 mg. capsules.

### Example 59. Example Four-Pulse Formulation

An example four-pulse formulation may be prepared as follows:

### Cannabinoid Matrix Pellet Formulation and Preparation Procedure

### Pellet Formulation

The composition of the cannabinoid matrix pellets provided in Table 12.

TABLE 12
Composition of Cannabinoid Pellets

| Component | Percentage (%) |
|---|---|
| Cannabinoid | 50 |
| Avicel PH 101 | 20 |
| Lactose | 20 |
| PVP K29/32* | 10 |
| Purified Water | |
| Total | 100 |

*PVP K29/32 was added as a 20% w/w aqueous solution during wet massing.

### Preparation Procedure for Cannabinoid Matrix Pellets

Blend cannabinoid and Avicel® PH 101 using a Robot Coupe high shear granulator.
Add 20% Povidone K29/32 binder solution slowly into the powder blend under continuous mixing.
Extrude the wet mass using an LCI Bench Top Granulator. The diameter of the screen of the Bench Top Granulator was 1.0 mm.
Spheronize the extrudate using a Model SPH20 Caleva Spheronizer.
Dry the spheronized pellets at 50.degree. C. overnight.
Pellets between 16 and 30 Mesh were collected for further processing.

### Preparation of an Eudragit® L30 D-55 Aqueous Coating Dispersion

### Dispersion Formulation

The composition of the aqueous Eudragit L30D-55 dispersion applied to the cannabinoid matrix pellets is provided below in Table 13.

TABLE 13
Eudragit. RTM. L 30 D-55 Aqueous Coating Dispersion

| Component | Percentage (%) |
|---|---|
| Eudragit. RTM. L 30 D-55 | 55.0 |
| Triethyl Citrate | 1.6 |
| Talc | 8.0 |
| Purified Water | 37.4 |
| Solids Content | 25.5 |
| Polymer Content | 15.9 |

### Preparation Procedure for an Eudragit® L30 D-55 Aqueous Dispersion

Suspend triethyl citrate and talc in deionized water.
The TEC/talc suspension is then homogenized using a PowerGen 700 high shear mixer.
Add the TEC/talc suspension slowly to the Eudragit® L30 D-55 latex dispersion while stirring.
Allow the coating dispersion to stir for one hour prior to application onto the cannabinoid matrix pellets.

### Preparation of an Eudragit® S 100 Aqueous Coating Dispersion

### Dispersion Formulation

The composition of the aqueous Eudragit® S 100 dispersion applied to the cannabinoid matrix pellets is provided below in Table 14.

TABLE 14
Eudragit. RTM. S 100 Aqueous Coating Dispersion

| | |
|---|---|
| Component | Percentage (%) |

Part A

| | |
|---|---|
| Eudragit. RTM. S 100 | 12.0 |
| 1N Ammonium Hydroxide | 6.1 |
| Triethyl Citrate | 6.0 |
| Purified Water | 65.9 |
| Part B Talc | 2.0 |
| Purified Water | 8.0 |
| Solid Content | 20.0 |
| Polymer Content | 12.0 |

### Preparation Procedure for an Eudragit® S 100 Aqueous Dispersion

### Part A:

Dispense Eudragit® S 100 powder in deionized water with stirring.
Add ammonium hydroxide solution drop-wise into the dispersion with stirring.
Allow the partially neutralized dispersion to stir for 60 minutes.
Add triethyl citrate drop-wise into the dispersion with stirring. Stir for about 2 hours prior to the addition of Part B.

### Part B:

Disperse talc in the required amount of water
Homogenize the dispersion using a PowerGen 700D high shear mixer.
Part B is then added slowly to the polymer dispersion in Part A with a mild stirring.

### Coating Conditions for the Application of Aqueous Coating Dispersions

The following coating parameters were used for coating with each of the Eudragit® L30 D-55 and Eudragit® S 100 aqueous film coatings.

### Coating Equipment

STREA 1™ Table Top Laboratory Fluid Bed Coater
Spray nozzle diameter 1.0 mm
Material Charge 300 gram
Inlet Air Temperature 40 to 45.degree. C.
Outlet Air Temperature 30 to 33.degree. C.
Atomization Air Pressure 1.8 Bar
Pump Rate 2 gram per minute 59.7.1
Coat matrix pellets with L30 D-55 dispersion such that you apply 12% coat weight gain to the pellets.
Coat matrix pellets with L30 D-55 dispersion such that you apply 30% coat weight gain to the pellets.
Coat matrix pellets with S100 dispersion such that you apply 20% coat weight gain to the pellets.

### Encapsulation of the Cannabinoid Pellets

Pellets are filled into size 00 hard gelatin capsules at a ratio of 20%:30%:20%:30% Immediate-release matrix pellets (uncoated), L30 D-55 coated pellets 12% weight gain, L30D-55 coated pellets 30% weight gain and S 100 coated pellets respectively.

The capsule is filled with the four different pellets to achieve a total dose of 375 mg/capsule.

### Example 60. Example Four-Pulse Formulation

An example four-pulse formulation may be prepared as follows:

### Cannabinoid Pellet Formulation and Preparation Procedure

### Pellet Formulations

The composition of the Cannabinoid matrix pellets provided in Table 15.

TABLE 15
Composition of Cannabinoid Matrix Pellets

| Component | Percentage (%) |
|---|---|
| Cannabinoid powder | 92 |
| Avicel PH 101 | 7.0 |
| Hydroxypropyl methylcellulose, NF* | 1.0 |
| Total | 100 |

*Hydroxypropyl methylcellulose was added as a 2.9% w/w aqueous solution during wet massing.

### Preparation Procedure for Cannabinoid Matrix Pellets

Blend Cannabinoid and Avicel® PH 101 using a low shear blender.
Add the hydroxypropyl methylcellulose binder solution slowly into the powder blend under continuous mixing.
Extrude the wet mass using an LCI Bench Top Granulator. The diameter of the screen of the Bench Top Granulator is 0.8 mm.
Spheronize the extrudate using a QJ-230 Spheronizer using a small cross section plate.
Dry the spheronized pellets at 60.degree. C. using a fluid bed dryer until the exhaust temperature reaches 40.degree. C.
Pellets between 20 and 40 Mesh were collected for further processing.

### Preparation of an Eudragit® L30 D-55 Aqueous Coating Dispersion

### Dispersion Formulation

The composition of the aqueous Eudragit L30D-55 dispersion applied to the cannabinoid matrix pellets is provided below in Table 16.

TABLE 16
Eudragit.RTM. L 30 D-55 Aqueous Coating Dispersion

| Component | Percentage (%) |
|---|---|
| Eudragit.RTM. L 30 D-55 | 41.6 |
| Triethyl Citrate | 2.5 |
| Talc | 5.0 |
| Purified Water | 50.9 |
| Solids Content | 20.0 |
| Polymer Content | 12.5 |

### Preparation Procedure for an Eudragit® L30 D-55 Aqueous Dispersion

Suspend triethyl citrate and talc in deionized water.
The TEC/talc suspension is mixed using laboratory mixer.
Add the TEC/talc suspension from slowly to the Eudragit® L30 D-55 latex dispersion while stirring.
Allow the coating dispersion to stir for one hour prior to application onto the cannabinoid matrix pellets.

### Preparation of an Eudragit® S 100 Aqueous Coating Dispersion

### Dispersion Formulation

The composition of the aqueous Eudragit® S 100 dispersion applied to the Cannabinoid matrix pellets is provided below in Table 17.

TABLE 17
Eudragit.RTM. S 100 Aqueous Coating Dispersion

| | |
|---|---|
| Component | Percentage (%) |

Part A

| | |
|---|---|
| Eudragit.RTM. S 100 | 10.0 |
| 1N Ammonium Hydroxide | 5.1 |
| Triethyl Citrate | 5.0 |
| Water | 64.9 |

Part B

| | |
|---|---|
| Talc | 2.0 |
| Water | 10.0 |
| Solid Content | 25.0 |
| Polymer Content | 10.0 |

### Preparation Procedure for an Eudragit® S 100 Aqueous Dispersion

### Part A:

Dispense Eudragit® S 100 powder in deionized water with stirring.
Add ammonium hydroxide solution drop-wise into the dispersion with stirring.
Allow the partially neutralized dispersion to stir for 60 minutes.
Add triethyl citrate drop-wise into the dispersion with stirring and let stir overnight prior to the addition of Part B.

### Part B:

Disperse talc in the required amount of water
Stir the dispersion using an overhead laboratory mixer.
Part B is then added slowly to the polymer dispersion in Part A with a mild stirring.

### Preparation of Aquacoat Coating Dispersion

### Dispersion Formulation

The composition of the aqueous Aquacoat dispersion applied to Cannabinoid L30 D-55 coated pellets is provided below in Table 18.

TABLE 18

| Component | Percentage (%) |
|---|---|
| Aquacoat ECD | 79.3 |
| Hydroxypropyl methylcellulose | 15.9 |
| Dibutyl Sebacate | 4.8 |
| Purified Water | (300 g) |

Prepare Hydroxypropyl methylcellulose (Methocel E15) solution by dispersing in water with continuous stirring. Add Aquacoat and dibutyl sebacate to the dispersion with stirring and continue to stir overnight.

### Coating Conditions for the Application of Aqueous Coating Dispersions

The following coating parameters were used for coating with each of the Eudragit® L30 D-55 and Eudragit® S 100 aqueous film coatings.

### Coating Equipment

STREA 1™ Table Top Laboratory Fluid Bed Coater
Spray nozzle diameter 1.0 mm
Material Charge 300 gram
Inlet Air Temperature 40 to 45.degree. C.
Outlet Air Temperature 30 to 33.degree. C.
Atomization Air Pressure 1.8 Bar
Pump Rate 2-6 gram per minute 60.9.1
Coat Cannabinoid matrix pellets with L30 D-55 dispersion to achieve a 20% coat weight gain.
Coat another batch of Cannabinoid matrix pellets with L30 D-55 dispersion to achieve a 20% weight gain.
Coat the L30 D-55 pellets with the Aquacoat Dispersion to achieve a 10% coat weight gain.
Coat Cannabinoid matrix pellets with S100 dispersion to achieve a 37% coat weight gain.

### Preparation of Cannabinoid Granulation for Tableting

TABLE 19
Composition of Cannabinoid Granulation (Immediate Release)

| Component | Percentage (%) |
|---|---|
| Cannabinoid powder | 92 |
| Avicel PH 101 | 7.0 |
| Hydroxypropyl methylcellulose, NF* | 1.0 |
| Total | 100 |

*Hydroxypropyl methylcellulose was added as a 2.9% w/w aqueous solution during wet massing.

Blend Cannabinoid and Avicel® PH 101 using a low shear blender.
Add the hydroxypropyl methylcellulose binder solution slowly into the powder blend under continuous mixing.
Dry the granulation at 60.degree. C. using a fluid bed dryer until the exhaust temperature reaches 40.degree. C.
Granules between 20 and 40 Mesh are collected for further processing.

### Tableting of the Cannabinoid Pellets

TABLE 20
Composition of Cannabinoid Tablets

| Component | Percentage (%) |
|---|---|
| Cannabinoid granules | 32.5 |
| Avicel PH 200 | 5.0 |
| Cannabinoid L30D-55 coated pellets | 20 |
| Cannabinoid Aquacoated pellets | 20 |
| Cannabinoid S100 coated pellets | 20 |
| Colloidal silicon dioxide | 1.5 |
| Magnesium stearate | 1.0 |
| Total | 100 |

Blend the Cannabinoid granules, Avicel PH-200, Cannabinoid pellets and colloidal silicon dioxide for 15 minutes in a tumble blender.
Add the magnesium stearate to the blender, and blend for 5 minutes.
Compress the blend on a rotary tablet press.
The fill weight should be adjusted to achieve a 500 mg dose tablet.

### Example 61. Example Four-Pulse Formulation

An example four-pulse formulation may be prepared as follows:

### Cannabinoid Pellet Formulation and Preparation Procedure

### Pellet Formulation

The composition of the cannabinoid matrix pellets provided in Table 21.

TABLE 21
Composition of Cannabinoid Pellets

| Component | Percentage (%) |
|---|---|
| Cannabinoid | 50.6 |
| Lactose monohydrate, spray dried | 32.1 |
| Silicified microcrystalline cellulose | 14.6 |
| Polyoxyl | 35 |
| Castor Oil* | 1.7 |
| Hydroxypropyl methylcellulose* | 1.0 |
| Total | 100 |

*Hydroxypropyl methylcellulose and Polyoxyl 35 were added as an 8.7% w/w aqueous solution during wet massing.

### Preparation Procedure for Cannabinoid Matrix Pellets

Blend cannabinoid, silicified microcrystalline cellulose and lactose monohydrate using a Robot Coupe high shear granulator.
Prepare the binder solution by adding the Polyoxyl to the purified water while stirring. After that is mixed, slowly add the hydroxypropyl methylcellulose and continue to stir until a solution is achieved.
Add binder solution slowly into the powder blend under continuous mixing.
Granulate the powders in the high shear granulator with the binder solution.
Extrude the wet mass using an LCI Bench Top Granulator. The diameter of the screen of the Bench Top Granulator was 1.2 mm.
Spheronize the extrudate using a Model SPH20 Caleva Spheronizer.
Dry the spheronized pellets at 50.degree. C. overnight.
Pellets between 18 and 30 Mesh were collected for further processing.

### Preparation of an Eudragit® L30 D-55 Aqueous Coating Dispersion

### Dispersion Formulation

The composition of the aqueous Eudragit L30D-55 dispersion applied to the cannabinoid matrix pellets is provided below in Table 22.

TABLE 22
Eudragit.RTM. L 30D-55 Aqueous Coating Dispersion

| Component | Percentage (%) |
|---|---|
| Eudragit.RTM. L 30D-55 | 40.4 |
| Triethyl Citrate | 1.8 |
| Talc | 6.1 |
| Water | 51.7 |
| Solids Content | 20.0 |
| Polymer Content | 12.1 |

### Preparation Procedure for an Eudragit® L30 D-55 Aqueous Dispersion

Suspend triethyl citrate and talc in deionized water.
The TEC/talc suspension is then homogenized using a PowerGen 700 high shear mixer.
Add the suspension slowly to the Eudragit® L30 D-55 latex dispersion while stirring.
Allow the coating dispersion to stir for one hour prior to application onto the cannabinoid matrix pellets.

### Preparation of an Eudragit® S 100 Aqueous Coating Dispersion

### Dispersion Formulation

The composition of the aqueous Eudragit® S 100 dispersion applied to the cannabinoid matrix pellets is provided below in Table 23.

TABLE 23
Eudragit.RTM. S 100 Aqueous Coating Dispersion

| | |
|---|---|
| Component | Percentage (%) |

Part A

| | |
|---|---|
| Eudragit.RTM. S 100 | 10.0 |
| 1N Ammonium Hydroxide | 5.1 |
| Triethyl Citrate | 5.0 |
| Water | 64.9 |

Part B

| | |
|---|---|
| Talc | 5.0 |
| Water | 10.0 |
| Solid Content | 25.0 |
| Polymer Content | 10.0 |

### Preparation Procedure for an Eudragit® S 100 Aqueous Dispersion

### Part A:

Dispense Eudragit® S 100 powder in deionized water with stirring.
Add ammonium hydroxide solution drop-wise into the dispersion with stirring.
Allow the partially neutralized dispersion to stir for 60 minutes
Add the triethyl citrate drop-wise to the dispersion and stir for 60 minutes prior to the addition of Part B.

### Part B:

Disperse talc in the required amount of water
Homogenize the dispersion using a PowerGen 700D high shear mixer.
Part B is then added slowly to the polymer dispersion in Part A with a mild stirring.

### Coating Conditions for the Application of Aqueous Coating Dispersions

The following coating parameters were used for coating with each of the Eudragit® L30 D-55 and Eudragit® S 100 aqueous film coatings.

### Coating Equipment

STREA 1™ Table Top Laboratory Fluid Bed Coater
Spray nozzle diameter 1.0 mm
Material Charge 300 gram
Inlet Air Temperature 40 to 45.degree. C.
Outlet Air Temperature 30 to 33.degree. C.
Atomization Air Pressure 1.6 Bar
Pump Rate 2 gram per minute
Coat matrix pellets with L30 D-55 dispersion such that you apply 12% coat weight gain to the pellets. Coat matrix pellets with L30 D-55 dispersion such that you apply 30% coat weight gain to the pellets. Coat matrix pellets with S100 dispersion such that you apply 37% coat weight gain to the pellets.

### Encapsulation of the Cannabinoid Pellets

Pellets are filled into size 00 hard gelatin capsules at a ratio of 20%:30%:20%:30% Immediate-release matrix pellets (uncoated), L30 D-55 coated pellets 12% weight gain, L30D-55 coated pellets 30% weight gain and S100 coated pellets respectively. The capsule is filled with the four different pellets to achieve a total dose of 250 mg/capsule.

### Example 62. Example Four-Pulse Formulation

An example four-pulse formulation may be prepared as follows:

### Cannabinoid Pellet Formulation and Preparation Procedure

### Pellet Formulations

The composition of the Cannabinoid pellets provided in Table 23.

TABLE 23
Composition of Cannabinoid Pellets

| Component | Percentage (%) |
|---|---|
| Cannabinoid powder | 92 |
| Avicel PH 101 | 6.0 |
| Polyoxyl | 35 |
| Castor Oil* | 1.0 |
| Hydroxypropyl methylcellulose, NF* | 1.0 |
| Purified Water ** | |
| Total | 100 |

*Hydroxypropyl methylcellulose and Cremaphor EL were added as a 2.9% w/w aqueous solution during wet massing.
** Removed during processing

### Preparation Procedure for Cannabinoid Pellets

Blend Cannabinoid and Avicel® PH 101 using a low shear blender. Add the hydroxypropyl methylcellulose and Polyoxyl 35 Castor Oil binder solution slowly into the powder blend under continuous mixing. Extrude the wet mass using an LCI Bench Top Granulator. The diameter of the screen of the Bench Top Granulator is 0.8 mm. Spheronize the extrudate using a QJ-230 Spheronizer using a small cross section plate. Dry the spheronized pellets at 60.degree. C. using a fluid bed dryer until the exhaust temperature reaches 40.degree. C. Pellets between 20 and 40 Mesh were collected for further processing.

### Cannabinoid Enteric-Release Pellet Formulation and Preparation Procedure

### Preparation of an Eudragit® L30 D-55/Eudragit NE 30D Aqueous Coating Dispersion

### Dispersion Formulation

The composition of the aqueous Eudragit L30D-55/Eudragit NE 30D aqueous coating dispersion applied to the cannabinoid pellets is provided below in Table 24.

TABLE 24
Eudragit.RTM. L 30D-55/Eudragit
NE 30D Aqueous Coating Dispersion

| Component | Percentage (%) |
|---|---|
| Eudragit.RTM. L 30D-55 | 44.4 |
| Eudragit NE 30D | 14.8 |
| Triethyl Citrate | 1.3 |
| Imwitor 900 | 0.9 |
| Purified Water* | 38.6 |
| Solid Content | 20.6 |
| Polymer Content | 16.4 |

*Removed during processing

### Preparation Procedure for an Eudragit® L30D-55/Eudragit NE 30D Aqueous Dispersion

Heat purified water to 75-80.degree. C. and then add triethyl citrate (TEC) and Imwitor 900. Homogenize dispersion until temperature is less than 55.degree. C. The TEC/Imwitor 900 dispersion is then stirred until the temperature is less than 35.degree. C. Add the TEC/Imwitor 900 dispersion to Eudragit L30D-55 latex dispersion and stir for at least 30 minutes. Add Eudragit NE 30D to the Eudragit L30D/TEC/Imwitor 900 dispersion and stir for at least 10 minutes. Screen the dispersion through a No. 60 mesh sieve prior to coating. Continue to stir the dispersion until the coating process is complete.

### Coating Conditions for the Application of Eudragit L30D-55/Eudragit NE 30D Aqueous Coating Dispersion

The following coating parameters were used for coating of the Eudragit® L30 D-55/Eudragit NE30D film coating dispersion.
Coating Equipment STREA 1™ Table Top Laboratory Fluid Bed Coater
Spray nozzle diameter 1.0 mm
Material Charge 300 gram
Inlet Air Temperature 45.degree. C.
Outlet Air Temperature 32 to 35.degree. C.
Atomization Air Pressure 1.6 Bar
Pump Rate 3-4 gram per minute
Coat Cannabinoid pellets with Eudragit L30 D-S5/Eudragit NE 30D film coating dispersion such that you apply 20% coat weight gain to the pellets.

### Cannabinoid Delayed Enteric-Release Pellets Formulation and Preparation Procedure

### Preparation of an AQOAT AS-HF Aqueous Coating Dispersion

### Dispersion Formulation

The composition of the aqueous AQOAT AS-HF aqueous coating dispersion applied to the cannabinoid pellets is provided below in Table 25.

TABLE 25
AQOAT AS-HF Aqueous Coating Dispersion

| Component | Percentage (%) |
|---|---|
| AQOAT AS-HF | 7.0 |
| Triethyl Citrate | 2.0 |
| Talc | 2.1 |
| Sodium lauryl sulfate | 0.2 |
| Purified Water* | 88.7 |
| Solid Content | 11.3 |
| Polymer Content | 7.0 |

*Removed during processing

### Preparation Procedure for an AQOAT AS-HF Aqueous Dispersion

Add triethyl citrate (TEC) to the purified water with stirring. Add the sodium lauryl sulfate (SLS) to the TEC dispersion with stirring and completely until completely dissolved. Add the AQOAT to the TEC/SLS dispersion and stir for at least 30 minutes. Add the talc to the AQOAT dispersion and until completely mixed and for at least 30 minutes. Screen the dispersion through a No. 60 mesh sieve prior to coating. Continue to stir the dispersion until the coating process is complete.

### Coating Conditions for the Application of AQOAT AS-HF Aqueous Coating Dispersion

The following coating parameters were used for coating of the AQOAT AS-HF film coating dispersion.
Coating Equipment STREA 1™ Table Top Laboratory Fluid Bed Coater
Spray nozzle diameter 1.0 mm
Material Charge 300 gram
Inlet Air Temperature 48.degree. C.
Outlet Air Temperature 27.degree. C.
Atomization Air Pressure 1.6 Bar
Pump Rate 3-4 gram per minute
Coat cannabinoid pellets with AQOAT AS-HF film coating dispersion such that you apply 30-35% coat weight gain to the pellets.

### Cannabinoid Colonic-Release Pellet Formulation and Preparation Procedure

### Preparation of an Eudragit® FS 30D Aqueous Coating Dispersion

### Dispersion Formulation

The composition of the aqueous Eudragit® FS 30D dispersion applied to the Cannabinoid pellets is provided below in Table 26.

TABLE 26
Eudragit.RTM. FS 30D Aqueous Coating Dispersion

| Component | Percentage (%) |
|---|---|
| Eudragit.RTM. FS 30D | 54.8 |
| Triethyl Citrate | 0.9 |
| Talc | 3.3 |
| Purified Water* | 41.0 |
| Solid Content | 20.6 |
| Polymer Content | 16.4 |

*Removed during processing

### Preparation Procedure for an Eudragit® FS 30D Aqueous Dispersion

Disperse triethyl citrate (TEC) in the purified water. Add the talc in the triethyl citrate dispersion.
Homogenize the dispersion using a homogenizer. Add slowly the Eudragit® FS 30D dispersion to the talc/TEC dispersion with stirring.
Continue to stir the coating dispersion until the coating process is complete.

### Coating Conditions for the Application of Eudragit FS30D Aqueous Coating Dispersion

The following coating parameters were used for coating with each of the Eudragit® FS 30 D aqueous film coating.
Coating Equipment STREA 1™ Table Top Laboratory Fluid Bed Coater
Spray nozzle diameter 1.2 mm
Material Charge 300 gram
Inlet Air Temperature 38.degree. C.
Outlet Air Temperature 22.degree. C.
Atomization Air Pressure 1.6 Bar
Pump Rate 6 gram per minute
Coat pellets with Eudragit FS 30D coating dispersion dispersion such that you apply 30% coat weight gain to the pellets.

### Cannabinoid Tablets

### Preparation of Cannabinoid Granulation for Tableting

TABLE 27
Composition of Cannabinoid Granulation (Immediate Release)

| Component | Percentage (%) |
|---|---|
| Cannabinoid powder | 92 |
| Avicel PH 101 | 7.0 |
| Hydroxypropyl methylcellulose, NF* | 1.0 |
| Total | 100 |

*Hydroxypropyl methylcellulose was added as a 2.9% w/w aqueous solution during wet massing.

Blend Cannabinoid and Avicel® PH 101 using a low shear blender. Add the hydroxypropyl methylcellulose binder solution slowly into the powder blend under continuous mixing. Dry the granulation at 60.degree. C. using a fluid bed dryer until the exhaust temperature reaches 40.degree. C. Granules between 20 and 40 Mesh are collected for further processing.

### Tableting of the Cannabinoid Pellets

TABLE 28
Composition of Cannabinoid Tablets

| Component | Percentage (%) |
|---|---|
| Cannabinoid granules | 32.5 |
| Avicel PH 200 | 5.0 |
| Eudragit L30D-55/NE 30D coated pellets | 20 |
| AQOAT coated pellets | 20 |
| Eudragit FS 30D coated pellets | 20 |
| Colloidal silicon dioxide | 1.5 |
| Magnesium stearate | 1.0 |
| Total | 100 |

Blend the Cannabinoid granules, Avicel PH-200, Cannabinoid coated pellets and colloidal silicon dioxide for 15 minutes in a tumble blender. Add the magnesium stearate to the blender, and blend for 5 minutes. Compress the blend on a rotary tablet press. The fill weight should be adjusted to achieve a 500 mg tablet.

### Encapsulation of the Cannabinoid Pellets

Pellets are filled into hard gelatin capsules at a ratio of 30%:30%:20%:20% Immediate-release pellets (uncoated), L30 D-55/Eudragit NE 30D coated pellets 20% weight gain, AQOAT coated pellets 30% weight gain and Eudragit FS 30D coated pellets respectively. The capsule is filled with the four different pellets to achieve a total dose of 250 mg/capsule.

### Example 63. Example Pellet Formulation and Preparation Procedure

An example Pellet Formulation and Preparation Procedure is as follows:

### Cannabinoid Pellet Formulation and Preparation Procedure

### Pellet Formulation

The composition of the Cannabinoid pellets provided in Table 29.

TABLE 29
Composition of Cannabinoid Pellets

| Component | Percentage (%) |
|---|---|
| Cannabinoid | 77.0 |
| Lactose monohydrate, spray dried | 11.0 |
| Croscarmellose sodium | 5.0 |
| Polyoxyl | 35 |
| Castor Oil* | 5.0 |
| Hydroxypropyl methylcellulose* | 2.0 |
| Purified water* | |
| Total | 100 |

*Removed during processing

### Preparation Procedure for Cannabinoid Pellets

Prepare the binder solution by adding the Polyoxyl to the purified water while stirring. After that is mixed, slowly add the hydroxypropyl methylcellulose and continue to stir until a solution is achieved. Blend Cannabinoid, lactose monohydrate, and croscarmellose sodium using a Robot Coupe high shear granulator. Add binder solution slowly into the powder blend under continuous mixing. Granulate the powders in the high shear granulator with the binder solution. Extrude the wet mass using an LCI Bench Top Granulator. The diameter of the screen of the Bench Top Granulator was 1.0 mm. Spheronize the extrudate using a Model SPH20 Caleva Spheronizer. Dry the spheronized pellets at 50.degree. C. until the moisture level is >3%. Pellets between 16 and 30 Mesh were collected for further processing.

### Cannabinoid Enteric-Release Pellet Formulation and Preparation Procedure

### Preparation of an Eudragit® L30 D-55/Eudragit NE 30D Aqueous Coating Dispersion

### Dispersion Formulation

The composition of the aqueous Eudragit L30D-55/Eudragit NE 30D aqueous coating dispersion applied to the Cannabinoid pellets is provided below in Table 30.

TABLE 30
Eudragit.RTM. L 30D-55/Eudragit
NE 30D Aqueous Coating Dispersion

| Component | Percentage (%) |
|---|---|
| Eudragit.RTM. L 30D-55 | 44.4 |
| Eudragit NE 30D | 14.8 |
| Triethyl Citrate | 1.3 |
| Imwitor 900 | 0.9 |
| Purified Water* | 38.6 |
| Solid Content | 20.6 |
| Polymer Content | 16.4 |

*Removed during processing

Preparation Procedure for an Eudragit® L30D-55/Eudragit NE 30D Aqueous Dispersion Heat purified water to 75-80.degree. C. and then add triethyl citrate (TEC) and Imwitor 900. Homogenize dispersion until temperature is less than 55.degree. C. The TEC/Imwitor 900 dispersion is then stirred until the temperature is less than 35.degree. C. Add the TEC/Imwitor 900 dispersion to Eudragit L30D-55 latex dispersion and stir for at least 30 minutes. Add Eudragit NE 30D to the Eudragit L30D/TEC/Imwitor 900 dispersion and stir for at least 10 minutes. Screen the dispersion through a No. 60 mesh sieve prior to coating. Continue to stir the dispersion until the coating process is complete.

### Coating Conditions for the Application of Eudragit L30D-55/Eudragit NE 30D Aqueous

### Coating Dispersion

The following coating parameters were used for coating of the Eudragit® L30 D-55/Eudragit NE30D film coating dispersion.
Coating Equipment STREA 1™ Table Top Laboratory Fluid Bed Coater
Spray nozzle diameter 1.0 mm
Material Charge 300 gram
Inlet Air Temperature
45.degree. C.
Outlet Air Temperature 32 to 35.degree. C.
Atomization Air Pressure 1.6 Bar
Pump Rate 3-4 gram per minute
Coat Cannabinoid pellets with Eudragit L30 D-55/Eudragit NE 30D film coating dispersion such that you apply 20% coat weight gain to the pellets.

### Cannabinoid Delayed Enteric-Release Pellets Formulation and Preparation Procedure

### Preparation of an AQOAT AS-HF Aqueous Coating Dispersion

### Dispersion Formulation

The composition of the aqueous AQOAT AS-HF aqueous coating dispersion applied to the Cannabinoid pellets is provided below in Table 31.

TABLE 31
AQOAT AS-HF Aqueous Coating Dispersion

| Component | Percentage (%) |
|---|---|
| AQOAT AS-HF | 7.0 |
| Triethyl Citrate | 2.0 |
| Talc | 2.1 |
| Sodium lauryl sulfate | 0.2 |
| Purified Water* | 88.7 |
| Solid Content | 11.3 |
| Polymer Content | 7.0 |

*Removed during processing

### Preparation Procedure for an AQOAT AS-HF Aqueous Dispersion

Add triethyl citrate (TEC) to the purified water with stirring. Add the sodium lauryl sulfate (SLS) to the TEC dispersion with stirring and completely until completely dissolved. Add the AQOAT to the TEC/SLS dispersion and stir for at least 30 minutes. Add the talc to the AQOAT dispersion and until completely mixed and for at least 30 minutes. Screen the dispersion through a No. 60 mesh sieve prior to coating. Continue to stir the dispersion until the coating process is complete.

### Coating Conditions for the Application of AQOAT AS-HF Aqueous Coating Dispersion

The following coating parameters were used for coating of the AQOAT AS-HF film coating dispersion.
Coating Equipment STREA 1™ Table Top Laboratory Fluid Bed Coater
Spray nozzle diameter 1.0 mm
Material Charge 300 gram
Inlet Air Temperature 48.degree. C.
Outlet Air Temperature 27.degree. C.
Atomization Air Pressure 1.6 Bar
Pump Rate 3-4 gram per minute
Coat Cannabinoid pellets with AQOAT AS-HF film coating dispersion such that you apply 30-35% coat weight gain to the pellets.

### Cannabinoid Colonic-Release Pellets Formulation and Preparation Procedure

### Preparation of an Eudragit® FS30D Aqueous Coating Dispersion

### Dispersion Formulation

The composition of the aqueous Eudragit® FS 30D dispersion applied to the Cannabinoid pellets is provided below in Table 32.

TABLE 32
Eudragit.RTM. FS 30D Aqueous Coating Dispersion

| Component | Percentage (%) |
|---|---|
| Eudragit.RTM. FS 30D | 54.8 |
| Triethyl Citrate | 0.9 |
| Talc | 3.3 |
| Purified Water* | 41.0 |
| Solid Content | 20.6 |
| Polymer Content | 16.4 |

*Removed during processing

### Preparation Procedure for an Eudragit® FS 30D Aqueous Dispersion

Disperse triethyl citrate (TEC) in the purified water. Add the talc in the triethyl citrate dispersion. Homogenize the dispersion using a homogenizer. Add slowly the Eudragit® FS 30D dispersion to the talc/TEC dispersion with stirring. Continue to stir the coating dispersion until the coating process is complete.

### Coating Conditions for the Application of Eudragit FS30D Aqueous Coating Dispersion

The following coating parameters were used for coating with each of the Eudragit® FS 30 D aqueous film coating.
Coating Equipment STREA 1™ Table Top Laboratory Fluid Bed Coater
Spray nozzle diameter 1.2 mm
Material Charge 300 gram
Inlet Air Temperature 38.degree. C.
Outlet Air Temperature 22.degree. C.
Atomization Air Pressure 1.6 Bar
Pump Rate 6 gram per minute
Coat pellets with Eudragit FS 30D coating dispersion dispersion such that you apply 30% coat weight gain to the pellets.

### Encapsulation of the Cannabinoid Pellets

Pellets are filled into hard gelatin capsules at a ratio of 25%:25%:25%:25% Immediate-Release Pellets (uncoated), Eudragit L30 D-55/Eudagit NE 30D coated pellets 20% weight gain, AQOAT AS-HF coated pellets 30-35% weight gain and Eudragit FS 30D coated pellets respectively.

The capsule is filled with the four different pellets to achieve a total dose of 250 mg/capsule.

### Tableting of the Cannabinoid Pellets

### Cannabinoid Tablet Formula

TABLE 33
Cannabinoid Tablet

| Component | Percentage (%) |
|---|---|
| Eudragit.RTM. L30D/NE 30D coated pellets | 20.0 |
| AQOAT AS-HF coated pellets | 20.0 |
| Eudargit FS 30D coated pellets | 20.0 |
| Emcocel | 24.5 |
| Cannabinoid | 12.5 |
| Povidone K30 | 2.0 |
| Magnesium stearate | 1.0 |
| Purified water * | |

* Removed during processing

### Preparation Procedure for a Cannabinoid Tablet

Blend all the components together except coated pellets and magnesium stearate for 10 minutes using a granulator. Granulate the blend with purified water. Screen the granulate through a No. 16 mesh sieve. Dry the screened granulate in a fluid bed dryer at 50-60.degree. C. until the moisture level is less than 3%. Add the dry granulate, coated pellets to a tumble blender and blend for 10 minutes. Add to the blend the magnesium stearate and blend an additional 3 minutes. Compress the blend on a rotary tablet press to achieve a dose of 500 mg.

### Example 64. Example Four-Pulse Formulation

An example four-pulse formulation may be prepared as follows:

### Cannabinoid Pellet Formulation and Preparation Procedure

### Pellet Formulation

The composition of the Cannabinoid pellets provided in Table 33.

TABLE 33
Composition of Cannabinoid Pellets

| Component | Percentage (%) |
|---|---|
| Cannabinoid | 77.0 |
| Lactose monohydrate, spray dried | 11.0 |
| Croscarmellose sodium | 5.0 |
| Polyoxyl | 35 |
| Castor Oil* | 5.0 |
| Hydroxypropyl methylcellulose* | 2.0 |
| Purified water* | |
| Total | 100 |

*Removed during processing

### Preparation Procedure for Cannabinoid Pellets

Prepare the binder solution by adding the Polyoxyl to the purified water while stirring. After that is mixed, slowly add the hydroxypropyl methylcellulose and continue to stir until a solution is achieved. Blend Cannabinoid, lactose monohydrate, and croscarmellose sodium using a Robot Coupe high shear granulator. Add binder solution slowly into the powder blend under continuous mixing. Granulate the powders in the high shear granulator with the binder solution. Extrude the wet mass using an LCI Bench Top Granulator. The diameter of the screen of the Bench Top Granulator was 1.0 mm. Spheronize the extrudate using a Model SPH20 Caleva Spheronizer. Dry the spheronized pellets at 50.degree. C. until the moisture level is >3%. Pellets between 16 and 30 Mesh were collected for further processing.

### Cannabinoid Enteric-Release Pellet Formulation and Preparation Procedure

### Preparation of an Eudragit® L30 D-55/Eudragit NE 30D Aqueous Coating Dispersion

### Dispersion Formulation

The composition of the aqueous Eudragit L30D-55/Eudragit NE 30D aqueous coating dispersion applied to the Cannabinoid pellets is provided below in Table 34.

TABLE 34
Eudragit.RTM. L 30D-55/Eudragit
NE 30D Aqueous Coating Dispersion

| Component | Percentage (%) |
|---|---|
| Eudragit.RTM. L 30D-55 | 44.4 |
| Eudragit NE 30D | 14.8 |
| Triethyl Citrate | 1.3 |
| Imwitor 900 | 0.9 |
| Purified Water* | 38.6 |
| Solid Content | 20.6 |
| Polymer Content | 16.4 |

*Removed during processing

Preparation Procedure for an Eudragit® L30D-55/Eudragit NE 30D Aqueous Dispersion Heat purified water to 75-80.degree. C. and then add triethyl citrate (TEC) and Imwitor 900. Homogenize dispersion until temperature is less than 55.degree. C. The TEC/Imwitor 900 dispersion is then stirred until the temperature is less than 35.degree. C. Add the TEC/Imwitor 900 dispersion to Eudragit L30D-55 latex dispersion and stir for at least 30 minutes. Add Eudragit NE 30D to the Eudragit L30D/TEC/Imwitor 900 dispersion and stir for at least 10 minutes. Screen the dispersion through a No. 60 mesh sieve prior to coating. Continue to stir the dispersion until the coating process is complete.

### Coating Conditions for the Application of Eudragit L30D-55/Eudragit NE 30D Aqueous

### Coating Dispersion

The following coating parameters were used for coating of the Eudragit® L30 D-55/Eudragit NE30D film coating dispersion.
Coating Equipment STREA 1™ Table Top Laboratory Fluid Bed Coater
Spray nozzle diameter 1.0 mm
Material Charge 300 gram
Inlet Air Temperature 45.degree. C.
Outlet Air Temperature 32 to 35.degree. C.
Atomization Air Pressure 1.6 Bar
Pump Rate 3-4 gram per minute
Coat Cannabinoid pellets with Eudragit L30 D-55/Eudragit NE 30D film coating dispersion such that you apply 20% coat weight gain to the pellets.

### Cannabinoid Delayed Enteric-Release Pellets Formulation and Preparation

### Procedure

### Preparation of an AQOAT AS-HF Aqueous Coating Dispersion

### Dispersion Formulation

The composition of the aqueous AQOAT AS-HF aqueous coating dispersion applied to the Cannabinoid pellets is provided below in Table 35.

TABLE 35
AQOAT AS-HF Aqueous Coating Dispersion

| Component | Percentage (%) |
|---|---|
| AQOAT AS-HF | 7.0 |
| Triethyl Citrate | 2.0 |
| Talc | 2.1 |
| Sodium lauryl sulfate | 0.2 |
| Purified Water* | 88.7 |
| Solid Content | 11.3 |
| Polymer Content | 7.0 |

*Removed during processing

### Preparation Procedure for an AQOAT AS-HF Aqueous Dispersion

Add triethyl citrate (TEC) to the purified water with stirring. Add the sodium lauryl sulfate (SLS) to the TEC dispersion with stirring and completely until completely dissolved. Add the AQOAT to the TEC/SLS dispersion and stir for at least 30 minutes. Add the talc to the AQOAT dispersion and until completely mixed and for at least 30 minutes. Screen the dispersion through a No. 60 mesh sieve prior to coating. Continue to stir the dispersion until the coating process is complete.

### Coating Conditions for the Application of AQOAT AS-HF Aqueous Coating Dispersion

The following coating parameters were used for coating of the AQOAT AS-HF film coating dispersion.
Coating Equipment STREA 1™ Table Top Laboratory Fluid Bed Coater
Spray nozzle diameter 1.0 mm
Material Charge 300 gram
Inlet Air Temperature 48.degree. C.
Outlet Air Temperature 27.degree. C.
Atomization Air Pressure 1.6 Bar
Pump Rate 3-4 gram per minute
Coat Cannabinoid pellets with AQOAT AS-HF film coating dispersion such that you apply 30-35% coat weight gain to the pellets.

### Cannabinoid Colonic-Release Pellets Formulation and Preparation

### Procedure

### Preparation of an Eudragit® FS30D Aqueous Coating Dispersion

### Dispersion Formulation

The composition of the aqueous Eudragit® FS 30D dispersion applied to the Cannabinoid pellets is provided below in Table 36.

TABLE 36
Eudragit.RTM. FS 30D Aqueous Coating Dispersion

| Component | Percentage (%) |
|---|---|
| Eudragit.RTM. FS 30D | 54.8 |
| Triethyl Citrate | 0.9 |
| Talc | 3.3 |
| Purified Water* | 41.0 |
| Solid Content | 20.6 |
| Polymer Content | 16.4 |

*Removed during processing

### Preparation Procedure for an Eudragit® FS 30D Aqueous Dispersion

Disperse triethyl citrate (TEC) in the purified water. Add the talc in the triethyl citrate dispersion. Homogenize the dispersion using a homogenizer. Add slowly the Eudragit® FS 30D dispersion to the talc/TEC dispersion with stirring. Continue to stir the coating dispersion until the coating process is complete.

### Coating Conditions for the Application of Eudragit FS30D Aqueous Coating Dispersion

The following coating parameters were used for coating with each of the Eudragit® FS 30 D aqueous film coating.
Coating Equipment STREA 1™ Table Top Laboratory Fluid Bed Coater
Spray nozzle diameter 1.2 mm
Material Charge 300 gram
Inlet Air Temperature 38.degree. C.
Outlet Air Temperature 22.degree. C.
Atomization Air Pressure 1.6 Bar
Pump Rate 6 gram per minute
Coat pellets with Eudragit FS 30D coating dispersion dispersion such that you apply 30% coat weight gain to the pellets.

### Encapsulation of the Cannabinoid Pellets

Pellets are filled into hard gelatin capsules at a ratio of 25%:25%:25%:25% Immediate-Release Pellets (uncoated), Eudragit L30 D-55/Eudagit NE 30D coated pellets 20% weight gain, AQOAT AS-HF coated pellets 30-35% weight gain and Eudragit FS 30D coated pellets respectively.

The capsule is filled with the four different pellets to achieve a total dose of 200 mg/capsule.

### Tableting of the Cannabinoid Pellets

### Cannabinoid Tablet Formula

TABLE 37
Cannabinoid Tablet

| Component | Percentage (%) |
|---|---|
| Eudragit.RTM. L30D/NE 30D coated pellets | 20.0 |
| AQOAT AS-HF coated pellets | 20.0 |
| Eudragit FS 30D coated pellets | 20.0 |
| Emcocel | 24.5 |
| Cannabinoid | 12.5 |
| Povidone K30 | 2.0 |
| Magnesium stearate | 1.0 |
| Purified water * | |

* Removed during processing

### Preparation Procedure for a Cannabinoid Tablet

Blend all the components together except coated pellets and magnesium stearate for 10 minutes using a granulator. Granulate the blend with purified water. Screen the granulate through a No. 16 mesh sieve. Dry the screened granulate in a fluid bed dryer at 50-60.degree. C. until the moisture level is less than 3%. Add the dry granulate, coated pellets to a tumble blender and blend for 10 minutes. Add to the blend the magnesium stearate and blend an additional 3 minutes. Compress the blend on a rotary tablet press to achieve a dose of 400 mg.

### Example 65. Example Pellet Formulation and Preparation Procedure

An example Pellet Formulation and Preparation Procedure is as follows:

### Cannabinoid Pellet Formulation and Preparation Procedure

### Pellet Formulations

The composition of the Cannabinoid pellets provided in Table 38.

TABLE 38
Composition of Cannabinoid Pellets

| Component | Percentage (%) |
|---|---|
| Cannabinoid | 92 |
| Avicel PH 101 | 6.0 |
| Polyoxyl | 35 |
| Castor Oil* | 1.0 |
| Hydroxypropyl methylcellulose, NF* | 1.0 |
| Purified Water ** | |
| Total | 100 |

*Hydroxypropyl methylcellulose and Polyoxyl 35 Castor Oil were added as a 2.9% w/w aqueous solution during wet massing.
** Removed during processing

### Preparation Procedure for Cannabinoid Pellets

Blend Cannabinoid and Avicel® PH 101 using a low shear blender. Add the hydroxypropyl methylcellulose and Polyoxyl 35 Castor Oil binder solution slowly into the powder blend under continuous mixing. Extrude the wet mass using an LCI Bench Top Granulator. The diameter of the screen of the Bench Top Granulator is 0.8 mm. Spheronize the extrudate using a QJ-230 Spheronizer using a small cross section plate. Dry the spheronized pellets at 60.degree. C. using a fluid bed dryer until the exhaust temperature reaches 40.degree. C. Pellets between 20 and 40 Mesh were collected for further processing.

### Cannabinoid Enteric-Release Pellet Formulation and Preparation Procedure

### Preparation of an Eudragit® L30 D-55/Eudragit NE 30D Aqueous Coating Dispersion

### Dispersion Formulation

The composition of the aqueous Eudragit L30D-55/Eudragit NE 30D aqueous coating dispersion applied to the Cannabinoid pellets is provided below in Table 39.

TABLE 39
Eudragit.RTM. L 30D-55/Eudragit
NE 30D Aqueous Coating Dispersion

| Component | Percentage (%) |
|---|---|
| Eudragit.RTM. L 30D-55 | 44.4 |
| Eudragit NE 30D | 14.8 |
| Triethyl Citrate | 1.3 |
| Imwitor 900 | 0.9 |
| Purified Water* | 38.6 |
| Solid Content | 20.6 |
| Polymer Content | 16.4 |

*Removed during processing

### Preparation Procedure for an Eudragit® L30D-55/Eudragit NE 30D Aqueous Dispersion

Heat purified water to 75-80.degree. C. and then add triethyl citrate (TEC) and Imwitor 900. Homogenize dispersion until temperature is less than 55.degree. C. The TEC/Imwitor 900 dispersion is then stirred until the temperature is less than 35.degree. C. Add the TEC/Imwitor 900 dispersion to Eudragit L30D-55 latex dispersion and stir for at least 30 minutes. Add Eudragit NE 30D to the Eudragit L30D/TEC/Imwitor 900 dispersion and stir for at least 10 minutes. Screen the dispersion through a No. 60 mesh sieve prior to coating. Continue to stir the dispersion until the coating process is complete.

### Coating Conditions for the Application of Eudragit L30D-55/Eudragit NE 30D Aqueous

### Coating Dispersion

The following coating parameters were used for coating of the Eudragit® L30 D-55/Eudragit NE30D film coating dispersion.
Coating Equipment STREA 1™ Table Top Laboratory Fluid Bed Coater
Spray nozzle diameter 1.0 mm
Material Charge 300 gram
Inlet Air Temperature 45.degree. C.
Outlet Air Temperature 32 to 35.degree. C.
Atomization Air Pressure 1.6 Bar
Pump Rate 3-4 gram per minute
Coat Cannabinoid pellets with Eudragit L30 D-55/Eudragit NE 30D film coating dispersion such that you apply 20% coat weight gain to the pellets.

### Cannabinoid Delayed Enteric-Release Pellets Formulation and Preparation

### Procedure

### Preparation of an AQOAT AS-HF Aqueous Coating Dispersion

### Dispersion Formulation

The composition of the aqueous AQOAT AS-HF aqueous coating dispersion applied to the Cannabinoid pellets is provided below in Table 40.

TABLE 40
AQOAT AS-HF Aqueous Coating Dispersion

| Component | Percentage (%) |
|---|---|
| AQOAT AS-HF | 7.0 |
| Triethyl Citrate | 2.0 |
| Talc | 2.1 |
| Sodium lauryl sulfate | 0.2 |
| Purified Water* | 88.7 |
| Solid Content | 11.3 |
| Polymer Content | 7.0 |

*Removed during processing

### Preparation Procedure for an AQOAT AS-HF Aqueous Dispersion

Add triethyl citrate (TEC) to the purified water with stirring. Add the sodium lauryl sulfate (SLS) to the TEC dispersion with stirring and completely until completely dissolved. Add the AQOAT to the TEC/SLS dispersion and stir for at least 30 minutes. Add the talc to the AQOAT dispersion and until completely mixed and for at least 30 minutes. Screen the dispersion through a No. 60 mesh sieve prior to coating. Continue to stir the dispersion until the coating process is complete.

### Coating Conditions for the Application of AQOAT AS-HF Aqueous Coating Dispersion

The following coating parameters were used for coating of the AQOAT AS-HF film coating dispersion.
Coating Equipment STREA 1™ Table Top Laboratory Fluid Bed Coater
Spray nozzle diameter 1.0 mm
Material Charge 300 gram
Inlet Air Temperature 48.degree. C.
Outlet Air Temperature 27.degree. C.
Atomization Air Pressure 1.6 Bar
Pump Rate 3-4 gram per minute
Coat Cannabinoid pellets with AQOAT AS-HF film coating dispersion such that you apply 30-35% coat weight gain to the pellets.

### Cannabinoid Colonic-Release Pellet Formulation and Preparation Procedure

### Preparation of an Eudragit® FS 30D Aqueous Coating Dispersion

### Dispersion Formulation

The composition of the aqueous Eudragit® FS 30D dispersion applied to the Cannabinoid pellets is provided below in Table 41.

TABLE 41
Eudragit.RTM. FS 30D Aqueous Coating Dispersion

| Component | Percentage (%) |
|---|---|
| Eudragit.RTM. FS 30D | 54.8 |
| Triethyl Citrate | 0.9 |
| Talc | 3.3 |
| Purified Water* | 41.0 |
| Solid Content | 20.6 |
| Polymer Content | 16.4 |

*Removed during processing

### Preparation Procedure for an Eudragit® FS 30D Aqueous Dispersion

Disperse triethyl citrate (TEC) in the purified water. Add the talc in the triethyl citrate dispersion. Homogenize the dispersion using a homogenizer. Add slowly the Eudragit® FS 30D dispersion to the talc/TEC dispersion with stirring.

Continue to stir the coating dispersion until the coating process is complete.

### Coating Conditions for the Application of Eudragit FS30D Aqueous Coating Dispersion

The following coating parameters were used for coating with each of the Eudragit® FS 30 D aqueous film coating.
Coating Equipment STREA 1™ Table Top Laboratory Fluid Bed Coater
Spray nozzle diameter 1.2 mm
Material Charge 300 gram
Inlet Air Temperature 38.degree. C.
Outlet Air Temperature 22.degree. C.
Atomization Air Pressure 1.6 Bar
Pump Rate 6 gram per minute
Coat pellets with Eudragit FS 30D coating dispersion dispersion such that you apply 30% coat weight gain to the pellets.

### Encapsulation of the Cannabinoid Pellets

Pellets are filled into hard gelatin capsules at a ratio of 25%:25%:25%:25% Immediate-release pellets (uncoated), L30 D-55/Eudragit NE 30D coated pellets 20% weight gain, AQOAT coated pellets 30% weight gain and Eudragit FS 30D coated pellets respectively. The capsule is filled with the four different pellets to achieve a total dose of 250 mg/capsule.

### Cannabinoid Tablets

### Preparation of Cannabinoid Granulation for Tableting

TABLE 42
Composition of Cannabinoid Granulation (Immediate Release)

| Component | Percentage (%) |
|---|---|
| Cannabinoid | 92 |
| Avicel PH 101 | 7.0 |
| Hydroxypropyl methylcellulose, NF* | 1.0 |
| Total | 100 |

*Hydroxypropyl methylcellulose was added as a 2.9% w/w aqueous solution during wet massing.

Blend Cannabinoid and Avicel® PH 101 using a low shear blender. Add the hydroxypropyl methylcellulose binder solution slowly into the powder blend under continuous mixing. Dry the granulation at 60 degrees C. using a fluid bed dryer until the exhaust temperature reaches 40 degrees C. Granules between 20 and 40 Mesh are collected for further processing.

### Tableting of the Cannabinoid Pellets

TABLE 43
Composition of Cannabinoid Tablets

| Component | Percentage (%) |
|---|---|
| Cannabinoid granules | 32.5 |
| Avicel PH 200 | 5.0 |
| Eudragit L30D-55/NE 30D coated pellets | 20 |
| AQOAT coated pellets | 20 |
| Eudragit FS 30D coated pellets | 20 |
| Colloidal silicon dioxide | 1.5 |
| Magnesium stearate | 1.0 |
| Total | 100 |

Blend the Cannabinoid granules, Avicel PH-200, Cannabinoid coated pellets and colloidal silicon dioxide for 15 minutes in a tumble blender. Add the magnesium stearate to the blender, and blend for 5 minutes. Compress the blend on a rotary tablet press. The fill weight should be adjusted to achieve a 500 mg dose tablet.

### Example 66. Example Oral Pulse-Release Dosage Form

This Example provides an example oral pulse-release dosage form, as detailed in the following Table:

| | | | Pulse 1 | Pulse 2 | Pulse 3 | |
|---|---|---|---|---|---|---|
| | Quality | | Content | Content | Content | Total |
| Component | Standard | Function | (mg) | (mg) | (mg) | (mg) |
| THC Oil¹ | In-house | Active | 1.98 | — | — | 1.98 |
| CBD Oil² | In-house | Active | — | 12.8 | 6.64 | 19.44 |
| CBG Oil³ | In-house | Active | — | — | 96.25 | 96.25 |
| Sugar Spheres | USP/NF, EP | Substrate | — | 80.9 | 173.19 | 254.09 |
| Microcrystalline | USP/NF, EP | Diluent/Excipient | 45.0 | 45.0 | 89.28 | 179.28 |
| Cellulose LP200 | | | | | | |
| Vivacoat | Manufacturer | Functional Film | — | 20.0 | 35.27 | 55.27 |
| | | Coat | | | | |
| Croscarmellose | USP/NF, EP, | Disintegrant | 15.0 | 7.5 | 7.5 | 30.00 |
| Sodium | JP | | | | | |
| Isomalt | FCC | Diluent/Excipient | 102.44 | — | — | 102.44 |
| Silicon dioxide | FCC | Glidant/flow aid | — | 1.25 | 2.5 | 3.75 |
| Magnesium Stearate | USP/NF, EP | Lubricant | 2.5 | 2.5 | 2.5 | 7.50 |

| | | | | |
|---|---|---|---|---|
| Total | 166.92 | 169.95 | 413.13 | 750.0 |

¹In the Table above, the THC Oil contains 1.5 mg of THC.
²In the Table above, the CBD Oil contains 18.3 mg of CBD.
³In the Table above, the CBG Oil contains 70 mg of CBG and 6.7 mg of CBD.

### Example 67. Clinical Study of the Effects on Gastrointestinal Symptoms of Twice-Daily Administration of a Pulse-Release Cannabinoid Oral Dosage Form

A study was conducted in 57 subject patient volunteers (23 male; 34 female) who had expressed concern with their gastrointestinal symptoms. The patients had previously been diagnosed with the following conditions:
22 patients: Irritable Bowel Syndrome
8 patients: Crohn's Disease
6 patients: GERD (Gastro-Esophogeal Reflux Disease)
3 patients: Ulcerative Colitis
2 patients: Irritable Bowel Disease
1 patients: Gall bladder removal
15 patients: other or no diagnosis

The patients were enrolled in a clinical study of the effects on their gastrointestinal symptoms of twice-daily administration of the example pulse-release cannabinoid oral dosage form described in Examples 66. Patients were administered with the example formulation twice daily for 30 days. Every 7 days, the patients were asked to complete a survey questionnaire. The patients provided answers to the survey questions as follows:
Survey Question 1 asked “Did the Product Have an Effect?”
Survey Question 2 asked “When Did You Notice the Effect (Post-Dose)?”

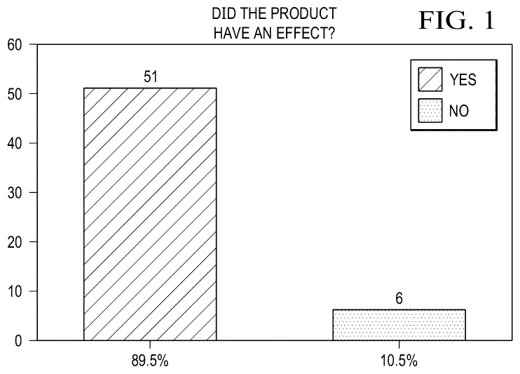

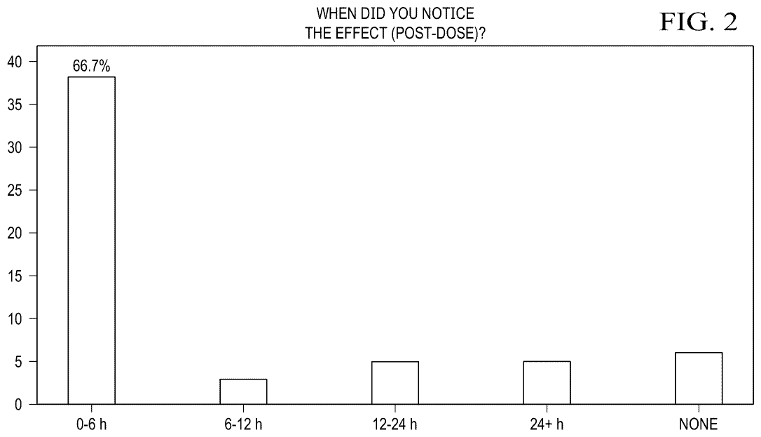

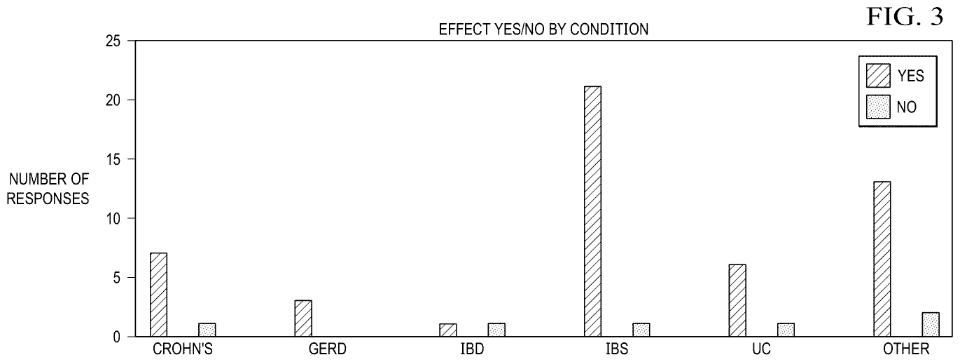

The survey answers to questions 1 and 2 above are shown respectively in FIG. 1 and FIG. 2. In addition, FIG. 3 shows the responses to question 1 broken out according to the patients' conditions.
Survey Question 3 asked “How long did the effects last?”

Following administration of the example formulation, the duration of effect of the example formulation on the patients' symptoms was up to 1 hour per day, up to 3 hours per day, up to 4 hours per day, up to 6 hours per day, from 6 to 12 hours per day, from 12 to 24 hours per day, or lasting longer than 24 hours. A delayed effect of treatment was observed in some patients, for example as follows:
Crohn's patient Male (born in 1983): No effect for 7 days; then a duration of effect of up to 6 hours per day consistently;
Crohn's patient Female (born in 1974): No effect for 3 days; then a duration of effect of 6-12 hours per day consistently;
Crohn's patient Male (born in 1950): No effect for 4 days; then a duration of effect of up to 6 hours per day consistently;
Crohn's patient Female (born in 1978): No effect for 3 days; then a duration of effect of up to 6 hours per day consistently;
IBD patient Female (born in 1954): No effect for 14 days; then a duration of effect of up to 6 hours per day consistently;
IBS patient Male (born in 1978): No effect for 3 days; then a duration of effect of up to 6 hours per day consistently;
IBS patient Female (born in 1979): No effect for 3 weeks; then a duration of effect of 24 hours or more post-dose consistently;
IBS patient Female (born in 1990): No effect for 5 days; then a duration of effect of 24 hours or more post-dose consistently;
IBS patient Male (born in 1958): No effect for 6 days; then a duration of effect of up to 6 hours per day consistently;
IBS patient Female (born in 1972): No effect for 6 days; then a duration of effect of 24 hours or more post-dose consistently;
IBS patient Male (born in 1983): No effect for 6 days; then a duration of effect of 24 hours or more post-dose consistently;
IBS patient Female (born in 1985): No effect for 6 days; then a duration of effect of 24 hours or more post-dose consistently.

The patients were also surveyed with the following questions:
Survey Question 4 asked “How Did This Make You Feel in General?”

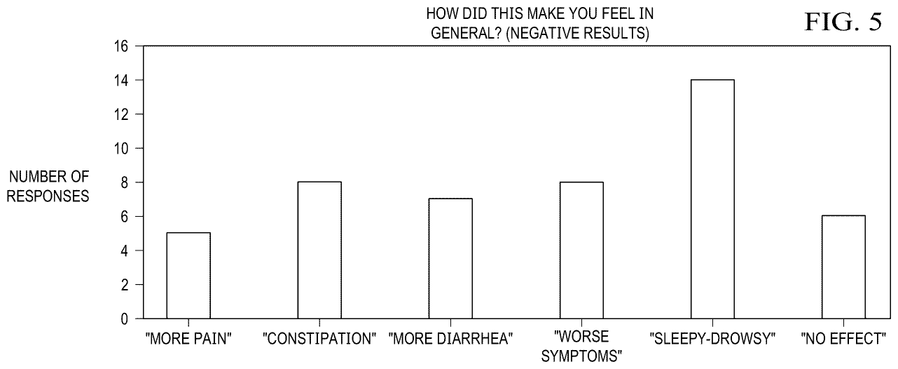

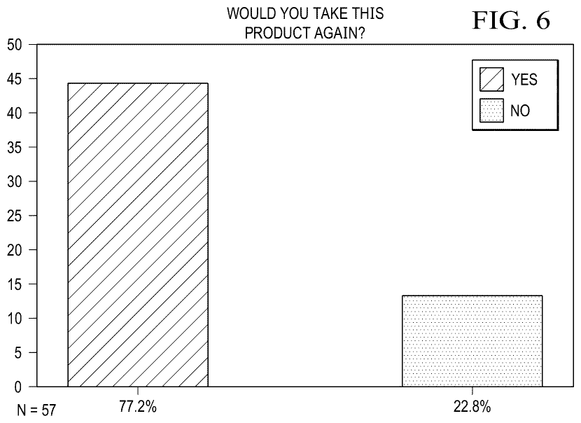

Positive results to question 4 are shown in FIG. 5, and negative results to question 4 are shown in FIG. 6. In general, most of the patients surveyed responded by stating they had positive results from twice daily administration with the pulse-release oral dosage form.
Survey Question 5 asked “Would You Take This Product Again?”

FIG. 7 shows the answers to Question 5, with over 77% of patients responding that they would take the pulse-release oral dosage form again.

Several patients commented on the breadth of symptoms addressed, with multiple types of different symptoms improved as a result of twice daily administration of the pulse-release oral dosage form. Such a result is surprising because, when treating patients with existing pharmaceutical products, typically administration with several different pharmaceutical products is needed to treat different symptoms such as diarrhea, bloating, cramps, pain, and so on. In addition, administration of the example pulse-release oral dosage form has shown a good safety profile to date, with 1 Crohn's patient being administered with the pulse-release oral dosage form for over 18 months, 1 ulcerative colitis patient being administered with the pulse-release oral dosage form for over 9 months, and 3 healthy volunteers being administered with the pulse-release oral dosage form for 30 days with metabolic function monitoring. All renal, hepatic and hematological data were normal from initial assessment through 30 days of drug administration.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative (e.g., “or”) herein is taken to mean either one or both or any combination thereof of the alternatives. The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. When particular values or compositions are provided in the instant disclosure, unless otherwise stated, the meaning of “about” or “approximately” should be assumed to be within an acceptable error range for that particular value or composition.

The term “administering”, “administered” and grammatical variants refers to the physical introduction of an agent to a subject, using any of the various methods and delivery systems known to those skilled in the art. Exemplary routes of administration include intravenous, intramuscular, subcutaneous, intraperitoneal, spinal or other parenteral routes of administration, for example by injection or infusion. The phrase “parenteral administration” means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Non-parenteral routes include oral, topical, epidermal or mucosal route of administration, for example, intranasally, vaginally, rectally, sublingually or topically. Administering can also be performed, for example, once, a plurality of times, and/or over one or more extended periods.

Throughout this application various publications, patents, and/or patent applications are referenced. The disclosures of the publications, patents and/or patent applications are hereby incorporated by reference in their entireties into this application in order to more fully describe the state of the art to which this disclosure pertains.

The above disclosure contains various examples of oral pulse-release compositions and dosage forms, and methods of treating inflammatory gastrointestinal disorders in a subject by administering the oral pulse-release compositions and dosage forms. Aspects of these various examples may all be combined with one another, even if not expressly combined in the present disclosure, unless they are clearly mutually exclusive.

The above disclosed subject matter is to be considered illustrative, and not restrictive, and the appended claims are intended to cover all such modifications, enhancements, and other implementations which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. An oral pulse-release dosage form comprising a total dose of one or more cannabinoid active pharmaceutical ingredients (API), wherein the oral pulse-release dosage form comprises:
a first pulse-release component (C₁) comprising a first portion (P₁) of the one or more cannabinoid API (API-P₁); and
a second pulse-release component (C₂) comprising a second portion (P₂) of the one or more cannabinoid API (API-P₂);
wherein the total dose of each of the one or more cannabinoid API is divided between the first portion (P₁) in the first pulse-release component (C₁) and the second portion (P₂) in the at least second pulse-release component (C₂); and
wherein when the pulse-release dosage form is placed in an aqueous solution of 0.1N HCl for two hours, followed by phosphate buffered solution at pH 6.0 and 37° C.±0.5° C. for four hours, followed by phosphate buffered solution at pH 7.2 and 37° C.±0.5° C. for up to fourteen hours, the pulse-release dosage form provides release of the one or more cannabinoid API-P₂ beginning from 1 to 6 hours after release of the API-P₁ begins.

2. The oral pulse-release dosage form of claim 1, further comprising a third pulse-release component (C₃) comprising a third portion (P₃) of the one or more cannabinoid API (API-P₃);
wherein the total dose of each of the one or more cannabinoid API is further divided between the first portion (P₁) in the first pulse-release component (C₁), the second portion (P₂) in the second pulse-release component (C₂), and the third portion (P₃) in the third pulse-release component (C₃); and
wherein when the pulse-release dosage form is placed in an aqueous solution of 0.1N HCl for two hours, followed by phosphate buffered solution at pH 6.0 and 37° C.±0.5° C. for four hours, followed by phosphate buffered solution at pH 7.2 and 37° C.±0.5° C. for up to fourteen hours, the pulse-release dosage form provides release of the one or more cannabinoid API-P₃ beginning from 1 to 6 hours after release of the API₁P₂ begins.

3. The oral pulse-release dosage form of claim 2, further comprising a fourth pulse-release component (C₄) comprising a fourth portion (P₄) of the one or more cannabinoid API (API-P₄);
wherein the total dose of each of the one or more cannabinoid API is further divided between the first portion (P₁) in the first pulse-release component (C₁), the second portion (P₂) in the second pulse-release component (C₂), the third portion (P₃) in the third pulse-release component (C₃), and the fourth portion (P₄) in the fourth pulse-release component (C₄); and
wherein when the pulse-release dosage form is placed in an aqueous solution of 0.1N HCl for two hours, followed by phosphate buffered solution at pH 6.0 and 37° C.±0.5° C. for four hours, followed by phosphate buffered solution at pH 7.2 and 37° C.±0.5° C. for up to fourteen hours, the pulse-release dosage form provides release of the one or more cannabinoid API-P₄ beginning from 1 to 6 hours after release of the API-P₃ begins.

4. The oral pulse-release dosage form of claim 3, further comprising a fifth pulse-release component (C₅) comprising a fifth portion (P₅) of the one or more cannabinoid API (API-P₅);
wherein the total dose of each of the one or more cannabinoid API is further divided between the first portion (P₁) in the first pulse-release component (C₁), the second portion (P₂) in the second pulse-release component (C₂), the third portion (P₃) in the third pulse-release component (C₃), the fourth portion (P₄) in the fourth pulse-release component (C₄), and the fifth portion (P₅) in the fifth pulse-release component (C₅); and
wherein when the pulse-release dosage form is placed in an aqueous solution of 0.1N HCl for two hours, followed by phosphate buffered solution at pH 6.0 and 37° C.±0.5° C. for four hours, followed by phosphate buffered solution at pH 7.2 and 37° C.±0.5° C. for up to fourteen hours, the pulse-release dosage form provides release of the one or more cannabinoid API-P₅ beginning from 1 to 6 hours after release of the API-P₄ begins.

5. The oral pulse-release dosage form of claim 1, wherein:
the pulse-release dosage form provides a second time of peak release rate (PRR₂) of each of the one or more cannabinoid API (PRR₂API) from about 1 to 6 hours after a first time of peak release rate (PRR₁) of each of the one or more cannabinoid API (PRR₁API).

6. The oral pulse-release dosage form of claim 5, wherein:
the PRR₁ is after 1, 2, 3, 4, 5, 6, 7, or 8 hours.

7. The oral pulse-release dosage form of claim 2, wherein:
the pulse-release dosage form provides a third time of peak release rate (PRR₃) of each of the one or more cannabinoid API (PRR₃API) from about 1 to 6 hours after a second time of peak release rate (PRR₂) of each of the one or more cannabinoid API (PRR₂API).

8. The oral pulse-release dosage form of claim 3, wherein:
the pulse-release dosage form provides a fourth time of peak release rate (PRR₄) of each of the one or more cannabinoid API (PRR₄API) from about 1 to 6 hours after a third time of peak release rate (PRR₃) of each of the one or more cannabinoid API (PRR₃API).

9. The oral pulse-release dosage form of claim 4, wherein:
the pulse-release dosage form provides a fifth time of peak release rate (PRR₅) of each of the one or more cannabinoid API (PRR₅API) from about 1 to 6 hours after a fourth time of peak release rate (PRR₄) of each of the one or more cannabinoid API (PRR₄API).

10. The oral pulse-release dosage form of any of claims 1-9, wherein the one or more cannabinoid API comprises cannabigerol (CBG).

11. The oral pulse-release dosage form of claim 10, wherein the total dose of CBG is up to 150 mg.

12. The oral pulse-release dosage form of claim 10, wherein the one or more cannabinoid API further comprises cannabidiol (CBD).

13. The oral pulse-release dosage form of claim 12, wherein the total dose of CBD is up to 40 mg.

14. The oral pulse-release dosage form of claim 12, wherein the one or more cannabinoid API further comprises delta-9-tetrahydrocannabinol (THC).

15. The oral pulse-release dosage form of claim 14, wherein the total dose of THC is up to 2 mg.

16. The oral pulse-release dosage form of any one of the preceding claims, wherein release of the total dose of the one or more cannabinoid API from the oral pulse-release dosage form occurs in up to 20 hours, up to 19 hours, up to 18 hours, up to 17 hours, up to 16 hours, up to 15 hours, up to 14 hours, up to 13 hours, up to 12 hours, up to 11 hours, up to 10 hours, up to 9 hours, up to 8 hours, or less.

17. The oral pulse-release dosage form of claim 1, wherein:
the first pulse-release component (C₁) is an immediate release (IR) formulation or a delayed-release (DR) formulation, wherein the first pulse-release component (C₁) comprises from 10% to 50% of the total dose independently of each of the one or more cannabinoid API; and
the second pulse-release component (C₂) is a delayed-release (DR) formulation comprising from 50% to 90% of the total dose independently of each of the one or more cannabinoid API.

18. The oral pulse-release dosage form of claim 2, wherein:
the first pulse-release component (C₁) is an immediate release (IR) formulation or a delayed-release (DR) formulation, wherein the first pulse-release component (C₁) comprises from 10% to 30% of the total dose independently of each of the one or more cannabinoid API; and
the second pulse-release component (C₂) and the third pulse-release component (C₃) are delayed-release (DR) formulations, wherein the second pulse-release component (C₂) comprises from 20% to 40% of the total dose independently of each of the one or more cannabinoid API, and the third pulse-release component (C₃) comprises from 30% to 70% of the total dose independently of each of the one or more cannabinoid API, such that together the second pulse-release component (C₂) and the third pulse-release component (C₃) comprise from 70% to 90% of the total dose independently of each of the one or more cannabinoid API.

19. The oral pulse-release dosage form of claim 3, wherein:
the first pulse-release component (C₁) is an immediate release (IR) formulation or a delayed-release (DR) formulation, wherein the first pulse-release component (C₁) comprises from 5% to 20% of the total dose independently of each of the one or more cannabinoid API; and
the second pulse-release component (C₂), the third pulse-release component (C₃), and the fourth pulse-release component (C₄) are delayed-release (DR) formulations, wherein the second pulse-release component (C₂) comprises from 15% to 30% of the total dose independently of each of the one or more cannabinoid API, the third pulse-release component (C₃) comprises from 20% to 40% of the total dose independently of each of the one or more cannabinoid API, and the fourth pulse-release component (C₄) comprises from 10% to 60% of the total dose independently of each of the one or more cannabinoid API, such that together the second pulse-release component (C₂), the third pulse-release component (C₃), and the fourth pulse-release component (C₄) comprise from 80% to 95% of the total dose independently of each of the one or more cannabinoid API.

20. The oral pulse-release dosage form of claim 4, wherein:
the first pulse-release component (C₁) is an immediate release (IR) formulation or a delayed-release (DR) formulation, wherein the first pulse-release component (C₁) comprises from 5% to 15% of the total dose independently of each of the one or more cannabinoid API; and
the second pulse-release component (C₂), the third pulse-release component (C₃), the fourth pulse-release component (C₄), and the fifth pulse-release component (C₅) are delayed-release (DR) formulations, wherein the second pulse-release component (C₂), comprises from 10% to 20% of the total dose independently of each of the one or more cannabinoid API, the third pulse-release component (C₃) comprises from 15% to 25% of the total dose independently of each of the one or more cannabinoid API, the fourth pulse-release component (C₄) comprises from 15% to 25% of the total dose independently of each of the one or more cannabinoid API, and the fifth pulse-release component (C₅) comprises from 15% to 55% of the total dose independently of each of the one or more cannabinoid API, such that together the second pulse-release component (C₂), the third pulse-release component (C₃), the fourth pulse-release component (C₄) and the fifth pulse-release component (C₅) comprise from 85% to 95% of the total dose independently of each of the one or more cannabinoid API.

21. The oral pulse-release dosage form of any one of claims 17 to 20, wherein the immediate release (IR) formulation comprises one or more pharmaceutically-acceptable excipients.

22. The oral pulse-release dosage form of claim 21, wherein the one or more pharmaceutically-acceptable excipient is selected from the group consisting of: microcrystalline cellulose, corn starch, pregelatinized starch, potato starch, rice starch, sodium carboxymethyl starch, hydroxypropylcellulose, hydroxypropylmethylcellulose, hydroxyethylcellulose, ethylcellulose, chitosan, hydroxychitosan, hydroxymethylatedchitosan, cross-linked chitosan, cross-linked hydroxymethyl chitosan, maltodextrin, mannitol, sorbitol, dextrose, maltose, fructose, glucose, levulose, sucrose, polyvinylpyrrolidone (PVP), an acrylic acid derivative (Carbopol, Eudragit, etc.), and a polyethylene glycol (PEG) such a low molecular weight PEGs (PEG2000-10000) and high molecular weight PEGs (Polyox) with molecular weights above 20,000 daltons, or any combination thereof.

23. The oral pulse-release dosage form of claim 21, wherein the pharmaceutically-acceptable excipients comprise from 1% to 60% by weight of the immediate release (IR) formulation.

24. The oral pulse-release dosage form of claim 21, wherein the immediate release (IR) formulation further comprises one or more pharmaceutically-acceptable surfactants.

25. The oral pulse-release dosage form of claim 24, wherein the one or more pharmaceutically-acceptable surfactant is selected from the group consisting of sodium lauryl sulfate, sodium monoglycerate, sorbitan monooleate, polyoxyethylene sorbitan monooleate, glyceryl monostearate, glyceryl monooleate, glyceryl monobutyrate, a non-ionic surfactant such as the Pluronic line of surfactants, and any other material with surface active properties.

26. The oral pulse-release dosage form of claim 24, wherein the pharmaceutically-acceptable surfactants comprise from 0.05% to 15% by weight of the immediate release (IR) formulation.

27. The oral pulse-release dosage form of any one of claims 17 to 20, wherein the delayed-release (DR) formulation comprises:
a core comprising the one or more cannabinoid API and one or more pharmaceutically-acceptable excipients and optionally one or more pharmaceutically-acceptable surfactants; and
delayed-release (DR) components comprising one or more polymers in an amount and configuration to delay release of the one or more cannabinoid API from the core.

28. The oral pulse-release dosage form of claim 27, wherein the one or more polymers of the delayed-release (DR) components are comprised in a layer coating the core, or are integrated within the core, or both.

29. The oral pulse-release dosage form of claim 27, wherein the delayed-release (DR) formulation is a non-pH sensitive delayed-release formulation, a pH sensitive delayed-release formulation, or a combination thereof.

30. The oral pulse-release dosage form of claim 29, wherein the one or more polymers of the delayed-release (DR) components of the non-pH sensitive delayed-release formulation comprise polyethylene glycol (PEG) with molecular weight above 4,000 daltons (Carbowax, Polyox), a wax such as white wax or bees wax, paraffin, an acrylic acid derivative (Eudragit), propylene glycol, ethylcellulose, or any combination thereof.

31. The oral pulse-release dosage form of claim 30, wherein the one or more polymers comprise from 0.05% to 25% by weight of the delayed-release (DR) formulation.

32. The oral pulse-release dosage form of claim 29, wherein the one or more polymers of the delayed-release (DR) components of the pH sensitive delayed-release formulation comprise cellulose acetate pthalate, Eudragit L, a pthalate salt of a cellulose derivative, or any combination thereof.

33. The oral pulse-release dosage form of claim 32, wherein the one or more polymers comprise from 4% to 25% by weight of the delayed-release (DR) formulation.

34. The oral pulse-release dosage form of any one of the preceding claims, wherein each of the pulse-release components is formulated as a plurality of particles or pellets.

35. The oral pulse-release dosage form of claim 34, wherein the particles or pellets are contained in a capsule, formed as a tablet, or suspended in a liquid.

36. A method of treating an inflammatory gastrointestinal disorder in a subject, the method comprising orally administering a therapeutically effective amount of the oral pulse-release dosage form of any of the preceding claims to the subject.

37. The method of claim 36, wherein the inflammatory gastrointestinal disorder comprises inflammation of the upper gastrointestinal tract, the lower gastrointestinal tract, or both.

38. The method of claim 36, wherein the administering is one, two, three or four times per day.

39. The method of claim 37, wherein the inflammation comprises inflammation of the upper gastrointestinal tract, and the first pulse-release component (C₁) is an immediate release (IR) formulation.

40. The method of claim 37, wherein the inflammation comprises inflammation of the lower gastrointestinal tract, and the first pulse-release component (C₁) is a delayed release (IR) formulation.

41. The method of claim 36, wherein the inflammatory gastrointestinal disorder comprises irritable bowel syndrome, inflammatory bowel disease, Crohn's disease, ulcerative colitis, perianal fistulizing disease (e.g., ulcerative colitis-related or Crohn's disease-related), left sided colitis, ulcerative proctitis, Gastro-Esophogeal Reflux Disease (GERD), an inflammatory gastrointestinal disorder following Gall bladder removal, an undiagnosed inflammatory gastrointestinal disorder, or any combination thereof.

42. The method of claim 36, wherein the administering results in treatment of the inflammatory gastrointestinal disorder within 6 hours post-dose, within 12 hours post-dose, within 24 hours post-dose, or greater than 24 hours post-dose.

43. The method of claim 36, wherein the administering results in treating one or more symptoms of the inflammatory gastrointestinal disorder for up to 1 hour per day, up to 2 hours per day, up to 3 hours per day, up to 4 hours per day, up to 5 hours per day, up to 6 hours per day, up to 12 hours per day, up to 24 hours per day, or for longer than 24 hours.

44. The method of claim 43, wherein the one or more symptoms comprises pain, bloating, diarrhea, loss of appetite, or any combination thereof.

45. The oral pulse-release dosage form of claim 2, wherein:
the first pulse-release component (C₁) is an immediate release (IR) formulation or a delayed-release (DR) formulation, wherein the first pulse-release component (C₁) comprises up to 10%, up to 20%, up to 30%, up to 40%, up to 50%, up to 60%, up to 70%, up to 80%, up to 90%, or up to 100% of the total dose independently of each of the one or more cannabinoid API; and
the second pulse-release component (C₂) and the third pulse-release component (C₃) are delayed-release (DR) formulations, wherein the second pulse-release component (C₂) comprises up to 10%, up to 20%, up to 30%, up to 40%, up to 50%, up to 60%, up to 70%, up to 80%, up to 90%, or up to 100% of the total dose independently of each of the one or more cannabinoid API, and the third pulse-release component (C₃) comprises up to 10%, up to 20%, up to 30%, up to 40%, up to 50%, up to 60%, up to 70%, up to 80%, up to 90%, or up to 100% of the total dose independently of each of the one or more cannabinoid API.

46. The oral pulse-release dosage form of claim 2, wherein:
the first pulse-release component (C₁) comprises up to 90%, up to 91%, up to 92%, up to 93%, up to 94%, up to 95%, up to 96%, up to 97%, up to 98%, up to 99%, or up to 100% of a total THC dose;
the third pulse-release component (C₃) comprises up to 90%, up to 91%, up to 92%, up to 93%, up to 94%, up to 95%, up to 96%, up to 97%, up to 98%, up to 99%, or up to 100% of a total CBG dose;
the second pulse-release component (C₂) comprises up to 60%, up to 61%, up to 62%, up to 63%, up to 64%, up to 65%, up to 66%, up to 67%, up to 68%, up to 69%, or up to 70% % of a total CBD dose; and
the third pulse-release component (C₃) comprises up to 30%, up to 31%, up to 32%, up to 33%, up to 34%, up to 35%, up to 36%, up to 37%, up to 38%, up to 39%, or up to 40% of the total CBD dose.
